SEQUENCE LISTING (28 sequences)

SEQ ID NO: 1 - protein, 4 aa, Artificial Sequence
  Synthetic
FALA

SEQ ID NO: 2 - protein, 5 aa, Artificial Sequence
  Synthetic
VLALA

SEQ ID NO: 3 - protein, 4 aa, Artificial Sequence
  Synthetic
ALAL

SEQ ID NO: 4 - protein, 5 aa, Artificial Sequence
  Synthetic
ALALA

SEQ ID NO: 5 - protein, 33 aa, Artificial Sequence
  Synthetic
LGPQGPPHLVADPSKKQGPWLEEEEEAYGWMDF

SEQ ID NO: 6 - protein, 4 aa, Artificial Sequence
  Synthetic
WXDF

SEQ ID NO: 7 - protein, 8 aa, Artificial Sequence
  Synthetic
DXMGWMDF

SEQ ID NO: 8 - protein, 8 aa, Artificial Sequence
  Synthetic
DXXGWXDF

SEQ ID NO: 9 - protein, 27 aa, Artificial Sequence
  Synthetic
VPLPAGGGTVLTKMYPRGNHWAVGHLM

SEQ ID NO: 10 - protein, 7 aa, Artificial Sequence
  Synthetic
WAVGHLM

SEQ ID NO: 11 - protein, 14 aa, Artificial Sequence
  Synthetic
AGCKNFFWKTFTSC

SEQ ID NO: 12 - protein, 8 aa, Artificial Sequence
  Synthetic
FCFWKTCT

SEQ ID NO: 13 - protein, 11 aa, Artificial Sequence
  Synthetic
RPLPQQFFGLM

SEQ ID NO: 14 - protein, 15 aa, Artificial Sequence
  Synthetic
PGTCEICAYAACTGC

SEQ ID NO: 15 - protein, 14 aa, Artificial Sequence
  Synthetic
NDDCELCVACTGCL

SEQ ID NO: 16 - protein, 16 aa, Artificial Sequence
  Synthetic
NYCCELCCNPACTGCF

SEQ ID NO: 17 - protein, 29 aa, Artificial Sequence
  Synthetic
HSDALFTDNYTRLRLQMAVKKYLNSILNG

SEQ ID NO: 18 - protein, 29 aa, Artificial Sequence
  Synthetic
HSDALFTDNYTRLRLQXAVKKYLNSILNG

SEQ ID NO: 19 - protein, 7 aa, Artificial Sequence
  Synthetic
AYGWXDF

SEQ ID NO: 20 - protein, 10 aa, Artificial Sequence
  Synthetic
EEEAYGWXDF

SEQ ID NO: 21 - protein, 5 aa, Artificial Sequence
  Synthetic
XLALA

SEQ ID NO: 22 - protein, 5 aa, Artificial Sequence
  Synthetic
XXLAL

SEQ ID NO: 23 - protein, 5 aa, Artificial Sequence
  Synthetic
XXLAL

SEQ ID NO: 24 - protein, 5 aa, Artificial Sequence
  Synthetic
XLALA

SEQ ID NO: 25 - protein, 15 aa, Artificial
  Synthetic
VLALAEEEAYGWXDF

SEQ ID NO: 26 - protein, 15 aa, Artificial
  Synthetic
VLALAEEEAYGWXDF

SEQ ID NO: 27 - protein, 15 aa, Artificial
  Synthetic
XLALAEEEAYGWXDF

SEQ ID NO: 28 - protein, 15 aa, Artificial
  Synthetic
FLALAEEEAYGWXDF

(19) United States
(12) Patent Application Publication
(10) Pub. No.: US 2005/0171014 A1
(43) Pub. Date: Aug. 4, 2005

(54) Conjugates of ligand linker and cytotoxic agent and related composition and methods of use

(75) Inventors: Nadya I Tarasova, Frederick, MD (US); Christopher J Michejda, Potomac, MD (US); Marcin Dyba, Frederick, MD (US); Carolyn Cohran, Frederick, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6780, US

(73) Assignee: Government of the United States of America, represented by the Secretary, Department of Health, Rockville, MD (US)

(21) Appl. No.: 10/505,239

(22) PCT Filed: Feb. 27, 2003
(86) PCT No.: PCT/US03/06344

(51) Int. Cl. A61K 38/17; C07K 14/595; C07K 14/655

(52) U.S. Cl. 514/12; 514/13; 530/324; 530/325; 530/311; 530/326

(57) ABSTRACT

A conjugate comprising a ligand, a linker, and a cytotoxic agent, in which the linker is FALA, VLALA, ALAL, ALALA, ChaLALA, ChaChaLAL, NalChaLAL or NalLALA; a composition thereof; a method of delivering a cytotoxic agent in a cell-specific manner; and a method of treating cancer in a mammal.

### FIELD OF THE INVENTION

This invention pertains to a conjugate comprising a ligand, a linker, and a cytotoxic agent, a composition thereof, a method of delivering a cytotoxic agent in a cell-specific manner, and a method of treating cancer.

### BACKGROUND OF THE INVENTION

Systemic toxicity of drugs is one of the most serious problems of cancer chemotherapy and frequently is dose limiting. The appearance of the various classes of multiple drug resistance renders even good drugs ineffective by expelling them from tumor cells (Ling, Cancer Chemother. Pharmacol. 40: Suppl, S3-S8 (1997)). Various strategies have been used to get around one or both of these difficulties, but they still are among the most intractable problems of cancer therapy. Targeting of drugs specifically to tumor cells has been the goal of many studies. Various protein toxins conjugated to monoclonal antibodies directed to specific tumor antigens have shown some promise as drugs (Pastan, Biochim. Biophys. Acta 1333: C1-C6 (1997)), but severe problems, such as the development of neutralizing antibodies (Chen et al., Gene Ther. 2: 116-123 (1995)), have limited the effectiveness of the method. Another promising approach is to use cellular receptors for growth factors (Kihara et al., Cancer Res. 55: 71-77 (1985); Carpenter, Curr. Opin. Cell Biol. 5: 261-264 (1993); Lemaristre et al., Breast Cancer Res. Treat. 32: 97-103 (1994)), cytokines (Strom et al., Annu. Rev. Med. 44: 343-353 (1993); Waldmann et al., Ann. Intern. Med. 116: 148-160 (1992)), or hormones (Roth et al., Anticancer Drug Des. 10: 655-666 (1994); Rink et al., Proc. Natl. Acad. Sci. 93: 15063-15068 (1996)) as targets to deliver cytotoxic moieties to the receptor-bearing cells. In this approach, the receptor binds to a ligand that is conjugated to a toxic moiety, resulting in receptor-mediated endocytosis, wherein the ligand-toxic moiety conjugate is internalized, along with the receptor, by the targeted cell. Once inside the cell, the conjugate is susceptible to lysosomal proteases that cleave the linkage between the ligand and toxin, resulting in the release of the toxin from the conjugate. Through this approach, the delivery of a drug to specific cell populations can be achieved.

There exists a need in the art for drug delivery conjugates, comprising a ligand, a linker, and a cytotoxic agent, that can deliver drugs to specific cell populations, such as cancer cells, and that can treat cancer through release of the cytotoxic agent. The present invention provides such drug conjugates. This and other objects of the invention, as well as additional inventive features, will be apparent from the description of the invention provided herein.

### BRIEF SUMMARY OF THE INVENTION

The present invention provides a conjugate comprising a ligand, a linker, and a cytotoxic agent, in which the linker is FALA (SEQ ID NO: 1), VLALA (SEQ ID NO: 2), or ChaLALA (SEQ ID NO: 21), ChaChaLAL (SEQ ID NO: 22), NalChaLAL (SEQ ID NO: 23) or NalLALA (SEQ ID NO: 24).

The present invention further provides a conjugate comprising a ligand, a linker and a cytotoxic agent, in which the linker is ALAL (SEQ ID NO: 3) and the ligand is LGPQGPPHLVADPSKKQGPWLEEEEEAYGWMDF (gastrin-34) (SEQ ID NO: 5), an N-terminal truncated derivative of gastrin-34, W(Nle)DF (SEQ ID NO: 6), D(SfY)MGWMDF (SEQ ID NO: 7), D(SfY)(Nle)GW(Nle)DF (SEQ ID NO: 8), EEEAYGW(Nle)DF (SEQ ID NO: 20), VPLPAGGGTVLTKMYPRGNHWAVGHLM (SEQ ID NO: 9), an N-terminal truncated derivative of VPLPAGGGTVLTKMYPRGNHWAVGHLM (SEQ ID NO: 9), WAVGHLM (SEQ ID NO: 10), AGCKNFFWKTFTSC (SEQ ID NO: 11), in which the two C residues are disulfide bonded, FCFWKTCT(OH) (SEQ ID NO: 12), in which the two C residues are disulfide bonded, RPLPQQFFGLM (SEQ ID NO: 13), an analog of RPLPQQFFGLM (SEQ ID NO: 13), PGTCEICAYAACTGC (SEQ ID NO: 14), in which the first and third C residues are disulfide bonded, PGTCEICAYAACTGC (SEQ ID NO: 14), in which the second and fourth C residues are disulfide bonded, NDDCELCVACTGCL (SEQ ID NO: 15), in which the first and third C residues are disulfide bonded, NDDCELCVACTGCL (SEQ ID NO: 15), in which the second and fourth C residues are disulfide bonded, NYCCELCCNPACTGCF (SEQ ID NO: 16), in which the first and fourth C residues are disulfide bonded, NYCCELCCNPACTGCF (SEQ ID NO: 16), in which the second and fifth C residues are disulfide bonded, NYCCELCCNPACTGCF (SEQ ID NO: 16), in which the third and sixth C residues are disulfide bonded, HSDALFTDNYTRLRLQMAVKKYLNSILNG (SEQ ID NO: 17), or HSDALFTDNYTRLRLQ(Nle)AVKKYLNSILNG (SEQ ID NO: 18).

The present invention also provides a conjugate comprising a ligand, a linker, and a cytotoxic drug, in which the linker is ALALA (SEQ ID NO: 4) and the ligand is gastrin-34, an N-terminal truncated derivative of gastrin-34 (provided that the derivative is not AYGW(Nle)DF (SEQ ID NO: 19)), W(Nle)DF (SEQ ID NO: 6), D(SfY)MGWMDF (SEQ ID NO: 7), D(SfY)(Nle)GW(Nle)DF (SEQ ID NO: 8), EEEAYGW(Nle)DF (SEQ ID NO: 20), VPLPAGGGTVLTKMYPRGNHWAVGHLM (SEQ ID NO: 9), an N-terminal truncated derivative of VPLPAGGGTVLTKMYPRGNHWAVGHLM (SEQ ID NO: 9), WAVGHLM (SEQ ID NO: 10), AGCKNFFWKTFTSC (SEQ ID NO: 11), in which the two C residues are disulfide bonded, FCFWKTCT(OH) (SEQ ID NO: 12), in which the two C residues are disulfide bonded, RPLPQQFFGLM (SEQ ID NO: 13), an analog of RPLPQQFFGLM (SEQ ID NO: 13), PGTCEICAYAACTGC (SEQ ID NO: 14), in which the first and third C residues are disulfide bonded, PGTCEICAYAACTGC (SEQ ID NO: 14), in which the second and fourth C residues are disulfide bonded, NDDCELCVACTGCL (SEQ ID NO: 15), in which the first and third C residues are disulfide bonded, NDDCELCVACTGCL (SEQ ID NO: 15), in which the second and fourth C residues are disulfide bonded, NYCCELCCNPACTGCF (SEQ ID NO: 16), in which the first and fourth C residues are disulfide bonded, NYCCELCCNPACTGCF (SEQ ID NO: 16), in which the second and fifth C residues are disulfide bonded, NYCCELCCNPACTGCF (SEQ ID NO: 16), in which the third and sixth C residues are disulfide bonded, HSDALFTDNYTRLRLQMAVKKYLNSILNG (SEQ ID NO: 17), or HSDALFTDNYTRLRLQ(Nle)AVKKYLNSILNG (SEQ ID NO: 18).

A composition comprising any of the above-described conjugates and a carrier is further provided by the present invention.

The present invention also provides a method of delivering a cytotoxic agent in a cell-specific manner. The method comprises administering any of the above-described conjugates to a collection of cells comprising a receptor to which the ligand of the conjugate binds.

Further provided by the present invention is a method of treating cancer in a mammal. The method comprises administering any of the above-described conjugates to the mammal.

### BRIEF DESCRIPTION OF THE DRAWINGS

FIG. 1 represents a table of amino acid sequences (N-terminal→C-terminal when read from left to right) of specific linkers and ligands of the conjugates of the present invention.

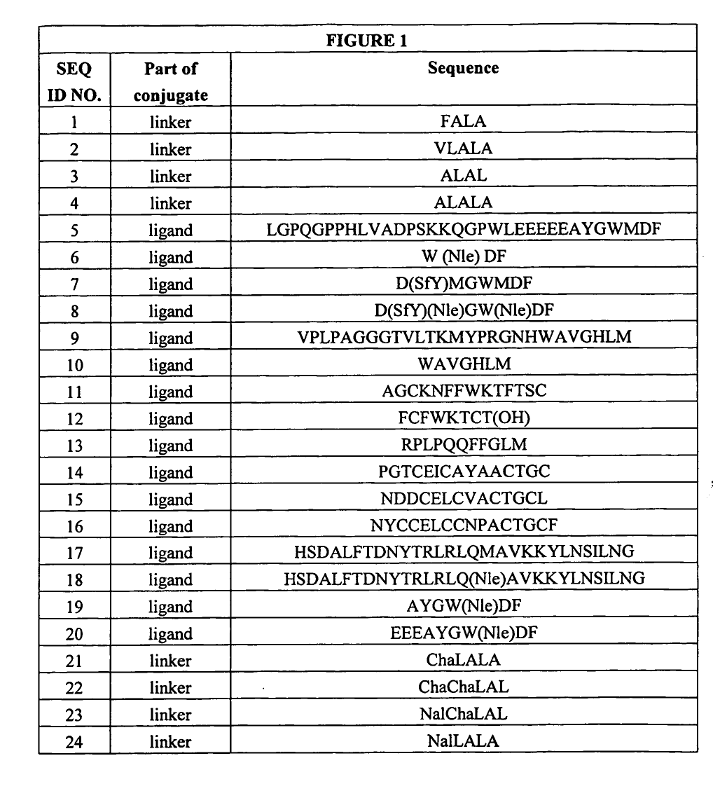

FIG. 2 represents a table of the ligands of the present invention and the receptors to which they specifically bind.

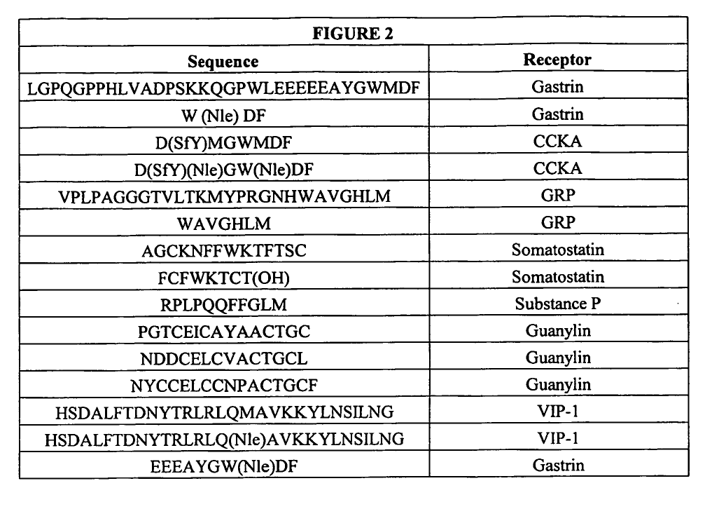

### DETAILED DESCRIPTION OF THE INVENTION

The present invention provides a conjugate comprising a ligand, a linker, and a cytotoxic agent, in which the linker is FALA (SEQ ID NO: 1), VLALA (SEQ ID NO: 2), ChaLALA (SEQ ID NO: 21), ChaChaLAL (SEQ ID NO: 22), NalChaLAL (SEQ ID NO: 23), or NalLALA (SEQ ID NO 24). “Cha” as used herein is an abbreviation for 2-cyclohexyl-L-alanine, whereas “Nal” is used herein as an abbreviation for 1-naphtyl-alanine.

With respect to the present invention, the ligand can be a peptide or a peptidomimetic. Desirably, the ligand comprises a functional group that can be attached to a linker and the attachment of the ligand to a linker does not eradicate the ability of the ligand to bind specifically to a cell-surface receptor. The term “peptide” as used herein means any polyamide that comprises two or more amino acids covalently linked by an amide bond between the carboxylic acid group of one and the alpha amino group of the other. It is generally appreciated by one skilled in the art that a peptide can optionally be glycosylated, amidated, carboxylated, phosphorylated, esterified, N-acetylated, or converted into an acid addition salt and/or optionally dimerized or polymerized. With respect to the present invention, the peptides are generally amidated unless otherwise indicated. The term “peptidomimetic” as used herein refers to a compound containing non-peptidic structural elements that is capable of mimicking or antagonizing the biological action(s) of a natural parent peptide. One skilled in the art will appreciate that a peptidomimetic does not have classical peptide characteristics, such as enzymatically scissille peptidic bonds. In one embodiment of the present invention, the peptidomimetic is a peptoid. The term “peptoid” as used herein refers to a peptidomimetic that results from the oligomeric assembly of N-substituted glycines. For example, CI-988, (see Augelli-Szafran et al., Bioorg. Med. Chem. 4: 1733-1745 (1996)), which has a carboxyl group (see arrow below) for attachment to a linker, can be a peptoid ligand of the conjugate of the present invention. 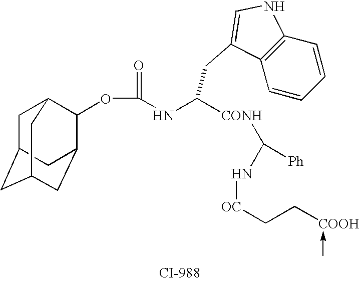
In contrast, L-365,260 cannot be a peptoid ligand of the conjugate of the present invention, since it lacks a functional group that can be used in attaching the linker.

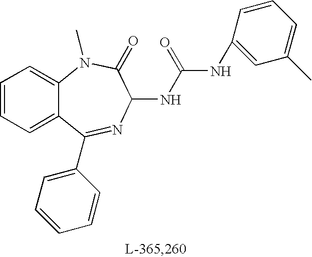

Desirably, the ligand specifically binds to a receptor. The term “specifically bind” as used herein refers to a ligand binding to a particular receptor over another receptor. The term “receptor” as used herein means a molecule or a polymeric structure in or on a cell that specifically recognizes and binds a compound that acts as a molecular messenger (i.e., a neurotransmitter, hormone, lymphokine, lectin, or drug). Examples of preferred ligands include those that bind to the gastrin receptor, the cholecystokinin A (CCKA) receptor, the somatostatin receptor, the gastrin-releasing peptide (GRP) receptor, the substance P receptor, the guanylin receptor, or the vasoactive intestinal peptide 1 (VIP-1) receptor.

More preferably, the ligand of the present inventive conjugate is LGPQGPPHLVADPSKKQGPWLEEEEEAYGWMDF (SEQ ID NO: 5) (also known in the art as gastrin-34), an N-terminal truncated derivative of gastrin-34, or W(Nle)DF (SEQ ID NO: 6). “Nle” as used herein is shorthand notation for “norleucine,” which is a non-naturally occurring analog of methionine that is more resistant to oxidation. The term “N-terminal truncated derivative” as used herein refers to a ligand that has the same amino acid sequence as another ligand but differs in that it has at least one amino acid deleted from its N-terminus. With respect to the present invention, it is preferred that the N-terminal truncated derivative of gastrin-34 comprises an amino acid sequence that is long enough to retain the ability to bind specifically to the receptor, yet short enough to retain the appropriate chemico-physical properties, such as solubility. It is more preferred that the N-terminal truncated derivative of gastrin-34 comprises the 7 most-C-terminal residues of gastrin-34, the 8-most-C-terminal residues of gastrin-34, the 9 most-C-terminal residues of gastrin-34, or the 10 most-C-terminal residues of gastrin-34. The above ligands specifically bind to the gastrin receptor, also known in the art as the cholecystokinin B (CCKB) receptor.

Alternatively, the ligand of the present inventive conjugate is D(SfY)MGWMDF (SEQ ID NO: 7), D(SfY)(Nle)GW(Nle)DF (SEQ ID NO: 8), or EEEAYGW(Nle)DF (SEQ ID NO: 20). “SfY” as used herein is an abbreviation for the modified amino acid sulfotyrosine. The ligands of SEQ ID NOS: 7 and 8 specifically bind to the CCKA receptor, whereas the ligand of SEQ ID NO: 20 specifically binds to the gastrin receptor. The ligand of the present conjugate also can be VPLPAGGGTVLTKMYPRGNHWAVGHLM (SEQ ID NO: 9), an N-terminal truncated derivative of VPLPAGGGTVLTKMYPRGNHWAVGHLM (SEQ ID NO: 9), or WAVGHLM (SEQ ID NO: 10). With respect to the present invention, it is preferred that the N-terminal truncated derivative of VPLPAGGGTVLTKMYPRGNHWAVGHLM comprises an amino acid sequence that is long enough to retain the ability to bind specifically to the receptor, yet short enough to retain the appropriate chemico-physical properties, such as solubility. It is more preferred that the N-terminal truncated derivative of VPLPAGGGTVLTKMYPRGNHWAVGHLM (SEQ ID NO: 9) includes, but is not limited to, the heptapeptide, WAVGHLM (SEQ ID NO. 10). Ligands of these amino acid sequences specifically bind to the GRP receptor, which is also known in the art as the bombesin receptor.

The ligand of the present inventive conjugate also can be AGCKNFFWKTFTSC (SEQ ID NO: 11), in which the two C residues are disulfide bonded, or FCFWKTCT(OH) (SEQ ID NO: 12), in which the two C residues are disulfide bonded. One skilled in the art will appreciate that (OH) represents a free carboxyl group at the end of the instant peptide. These ligands specifically bind to the somatostatin receptor. Yet another alternative ligand of the present invention is RPLPQQFFGLM (SEQ ID NO: 13) or an analog of RPLPQQFFGLM (SEQ ID NO: 13). By “analog” it is meant that the ligand is at least about about 70% (or 75%, 80%, 85%, 90% or 95%) identical to the parent ligand. These ligands specifically bind to the substance P receptor, which is also known in the art as the neurokinin 1 (NK1) receptor.

The ligand of the present invention also can be PGTCEICAYAACTGC (SEQ ID NO: 14), in which the first and third C residues are disulfide bonded, or GTCEICAYAACTGC (SEQ ID NO: 14), in which the second and fourth C residues are disulfide bonded. These ligands specifically bind to the guanylin receptor. Alternatively, the ligand can be NDDCELCVACTGCL (SEQ ID NO: 15), in which the first and third C residues are disulfide bonded, or NDDCELCVACTGCL (SEQ ID NO: 15), in which the second and fourth C residues are disulfide bonded. Also, the ligand of the conjugate can be NYCCELCCNPACTGCF (SEQ ID NO: 16), in which the first and fourth C residues are disulfide bonded, NYCCELCCNPACTGCF (SEQ ID NO: 16), in which the second and fifth C residues are disulfide bonded, or NYCCELCCNPACTGCF (SEQ ID NO: 16), in which the third and sixth C residues are disulfide bonded. These ligands specifically bind to the guanylin receptor. Finally, the ligand of the present inventive conjugate can be either HSDALFTDNYTRLRLQMAVKKYLNSILNG (SEQ ID NO: 17) or HSDALFTDNYTRLRLQ(Nle)AVKKYLNSILNG (SEQ ID NO: 18). These ligands specifically bind to the VIP-1 receptor.

The cytotoxic agent in the conjugate can be any agent known in the art but, preferably, the cytotoxic agent is cemadotin, a derivative of cemadotin, a derivative of hemiasterlin, esperamicin C, neocarzinostatin, maytansinoid DM1,7-chloromethyl-10,11 methylenedioxy-camptothecin, rhizoxin, or the halichondrin B analog ER-086526. The term “derivative” as used herein refers to a molecule that contains the same backbone structure of the parent molecule but is modified to some extent in the side-chains of the molecule. Some of these cytotoxic agents can be obtained by synthesizing them according to procedures that are described previously. (See, for example, Haupt et al., U.S. Pat. No. 5,831,002, for the synthesis of cemadotin and of cemadotin derivatives; see, for instance, Example 2 for the synthesis of hemiasterlin; see, for example, Lam et al., J. Antibiot. (Tokyo), 48(12): 1497-1501 (1995) for the synthesis of esperamicin C; see, for instance, Toshima et al., Angew. Chem. Int. Ed. Engl. 39(20): 3656-3658 (2000) for the synthesis of a neocarzinostatin chromophore; see, for example, Chari et al., International Patent Application No. WO 02/16368, for the synthesis of a maytansinoid DM1 derivative; see, for example, Lackey et al., U.S. Pat. No. 5,342,947 for the synthesis of 7-chloromethyl-10,11 methylenedioxy-camptothecin; see, for instance, Mitchell et al., Tetrahed. Lett. 43: 493-497 (2002) for the synthesis of rhizoxin; and see, for example, Littlefield et al., U.S. Pat. No. 6,214,865 for the synthesis of the halichrondrin analog, ER-086526. Alternatively, some of the cytotoxic agents can be purchased from companies, such as ImmunoGen Corp., which sells maytansinoid DM1, and Eisai Co., which sells the halichondrin B analog ER-086526.

The present invention further provides a conjugate comprising a ligand, a linker and a cytotoxic agent, in which the linker is ALAL (SEQ ID NO: 3) and the ligand is gastrin-34, an N-terminal truncated derivative of gastrin-34, W(Nle)DF (SEQ ID NO: 6), D(SfY)MGWMDF (SEQ ID NO: 7), D(SfY)(Nle)GW(Nle)DF (SEQ ID NO: 8), EEEAYGW(Nle)DF (SEQ ID NO: 20), VPLPAGGGTVLTKMYPRGNHWAVGHLM (SEQ ID NO: 9), an N-terminal truncated derivative of VPLPAGGGTVLTKMYPRGNHWAVGHLM (SEQ ID NO: 9), WAVGHLM (SEQ ID NO: 10), AGCKNFFWKTFTSC (SEQ ID NO: 11), in which the two C residues are disulfide bonded, FCFWKTCT(OH) (SEQ ID NO: 12), in which the two C residues are disulfide bonded, RPLPQQFFGLM (SEQ ID NO: 13), an analog of RPLPQQFFGLM (SEQ ID NO: 13), PGTCEICAYAACTGC (SEQ ID NO: 14), in which the first and third C residues are disulfide bonded, PGTCEICAYAACTGC (SEQ ID NO: 14), in which the second and fourth C residues are disulfide bonded, NDDCELCVACTGCL (SEQ ID NO: 15), in which the first and third C residues are disulfide bonded, NDDCELCVACTGCL (SEQ ID NO: 15), in which the second and fourth C residues are disulfide bonded, NYCCELCCNPACTGCF (SEQ ID NO: 16), in which the first and fourth C residues are disulfide bonded, NYCCELCCNPACTGCF (SEQ ID NO: 16), in which the second and fifth C residues are disulfide bonded, NYCCELCCNPACTGCF (SEQ ID NO: 16), in which the third and sixth C residues are disulfide bonded, HSDALFTDNYTRLRLQMAVKKYLNSILNG (SEQ ID NO: 17), or HSDALFTDNYTRLRLQ(Nle)AVKKYLNSILNG (SEQ ID NO: 18). The cytotoxic agent in the conjugate can be any agent known in the art but, preferably, the cytotoxic agent is cemadotin, a derivative of cemadotin, a derivative of hemiasterlin, esperamicin C, neocarzinostatin, maytansinoid DM1,7-chloromethyl-10,11 methylenedioxy-camptothecin, rhizoxin, or the halichondrin B analog ER-086526.

The present invention also provides a conjugate comprising a ligand, a linker, and a cytotoxic agent, in which the linker is ALALA (SEQ ID NO: 4) and ligand is gastrin-34, an N-terminal truncated derivative of gastrin-34 (provided that the derivative is not AYGW(Nle)DF (SEQ ID NO: 19)), W(Nle)DF (SEQ ID NO: 6), D(SfY)MGWMDF (SEQ ID NO: 7), D(SfY)(Nle)GW(Nle)DF (SEQ ID NO: 8), EEEAYGW(Nle)DF (SEQ ID NO: 20), VPLPAGGGTVLTKMYPRGNHWAVGHLM (SEQ ID NO: 9), an N-terminal truncated derivative of VPLPAGGGTVLTKMYPRGNHWAVGHLM (SEQ ID NO: 9), WAVGHLM (SEQ ID NO: 10). AGCKNFFWKTFTSC (SEQ ID NO: 11), in which the two C residues are disulfide bonded, FCFWKTCT(OH) (SEQ ID NO: 12), in which the two C residues are disulfide bonded, RPLPQQFFGLM (SEQ ID NO: 13), an analog of RPLPQQFFGLM (SEQ ID NO: 13), PGTCEICAYAACTGC (SEQ ID NO: 14), in which the first and third C residues are disulfide bonded, or PGTCEICAYAACTGC (SEQ ID NO: 14), in which the second and fourth C residues are disulfide bonded, NDDCELCVACTGCL (SEQ ID NO: 15), in which the first and third C residues are disulfide bonded, NDDCELCVACTGCL (SEQ ID NO: 15), in which the second and fourth C residues are disulfide bonded, NYCCELCCNPACTGCF (SEQ ID NO: 16), in which the first and fourth C residues are disulfide bonded, NYCCELCCNPACTGCF (SEQ ID NO: 16), in which the second and fifth C residues are disulfide bonded, NYCCELCCNPACTGCF (SEQ ID NO: 16), in which the third and sixth C residues are disulfide bonded, HSDALFTDNYTRLRLQMAVKKYLNSILNG (SEQ ID NO: 17), or HSDALFTDNYTRLRLQ(Nle)AVKKYLNSILNG (SEQ ID NO: 18). The cytotoxic agent in the conjugate can be any agent known in the art but preferably, the cytotoxic agent is cemadotin, a derivative of cemadotin, a derivative of hemiasterlin, esperamicin C, neocarzinostatin, maytansinoid DM1,7-chloromethyl-10,11 methylenedioxy-camptothecin, rhizoxin, or the halichondrin B analog ER-086526.

The conjugates of the present invention can be made by a variety of methods. It is preferred that, in general, the linker, which can be synthesized through use of an automated peptide synthesizer, is first attached to the ligand through a method that is dependent upon the type of ligand. If the ligand is a peptide, then the ligand and linker can be synthesized as one long peptide through use of an automated peptide synthesizer. If the ligand is a peptidomimetic, then the linker will have to attach to a functional group of the peptidomimetic. Preferred functional groups of the peptidomimetic include carboxyl, amino, sulfhydryl, and hydroxyl functional groups. CI-338, is, for example, a preferred peptidomimetic, since it contains a carboxyl group that can used to attach the linker to the ligand. In contrast, L-365,260 is not a preferred peptidomimetic to be used in the present invention, as it does not contain a preferred functional group useful for attaching a linker. However, one skilled in the art will appreciate that such peptidomimetics, which lack functional groups suitable for attaching the linker, could be modified such that they do contain an appropriate functional group for attachment of the linker. It is preferred that such a modification does not adversely affect the ability of the peptidomimetic to bind to the receptor nor significantly alter other chemico-physical properties of the peptidomimetic.

Once the ligand is attached to the linker, the cytotoxic agent then can be attached to the ligand-linker construct. The method by which the cytotoxic agent is attached depends upon the type of functional group on the agent that is available for attaching the ligand-linker construct. Preferably, the cytotoxic agent contains a carboxyl group to which the ligand-linker construct can be attached. Alternatively, if the functional group of the cytotoxic agent is a hydroxyl or an amino functional group, then it is preferred that succinate anhydride be used to reverse the polarity of the ligand-linker construct, such that a carboxyl group of succinate can be used to attach the ligand-linker construct to the cytotoxic agent. For exemplary methods of synthesizing the conjugates of the present invention, see Example 1 and Example 2 of the instant patent application.

The present invention further provides a composition comprising any of the above-described conjugates and a carrier. Preferably, the carrier is pharmaceutically acceptable. With respect to the conjugates of the present invention, the carrier can be any of those conventionally used and is limited only by chemico-physical considerations, such as solubility and lack of reactivity with the conjugates of the present invention, and by the route of administration. It is preferred that the pharmaceutically acceptable carrier be one which is chemically inert to the conjugates and one which has no detrimental side effects or toxicity under the conditions of use. The pharmaceutically acceptable carriers described herein, for example, vehicles, adjuvants, excipients, and diluents, are well-known to those skilled in the art and are readily available to the public. Typically, the composition, such as a pharmaceutical composition, comprising the carrier and the conjugate of the present invention can comprise a physiological saline solution; dextrose or other saccharide solution; or ethylene, propylene, polyethylene, or other glycol.

The present invention also provides a method of delivering a cytotoxic agent in a cell-specific manner. The method comprises administering any of the above-described conjugates or compositions to a collection of cells comprising a receptor to which the ligand of the conjugate binds, whereupon the cytotoxic agent is administered to the cells in a cell-specific manner. With respect to the present inventive method, the term “cell-specific manner” as used herein refers to the delivery of the cytotoxic agent being selective for one cell over another. The cells can be any cells, but, preferably, they are in vivo.

Further provided by the present invention is a method of treating cancer in a mammal. The method comprises administering to the mammal a cancer-treating effective amount of an above-described conjugate or composition, whereupon the cancer in the mammal is treated. A “cancer-treating effective amount” is an amount sufficient to treat existing cancer to any degree or to inhibit the onset of cancer. The cancer can be cancer of any tissue of a mammal, but, preferably, the cancer is cancer of the lung, stomach, colon, breast, or pancreas.

For purposes of the present invention, mammals include, but are not limited to, the order Rodentia, such as mice, and the order Logomorpha, such as rabbits, the order Carnivora, including Felines (cats) and Canines (dogs), the order Artiodactyla, including Bovines (cows) and Suines (pigs), the order Perssodactyla, including Equines (horses), the order Primate, Ceboid, or Simoid (monkeys), or the order Anthropoids (humans and apes). An especially preferred mammal is the human.

The dose administered to an animal, particularly a human, in the context of the present invention should be sufficient to effect a therapeutic response in the animal over a reasonable time frame. The dose will be determined by the strength of the particular conjugate or composition and the condition of the animal (e.g., human), as well as the body weight of the animal (e.g., human) to be treated. The size of the dose also will be determined by the existence, nature, and extent of any adverse side effects that might accompany the administration of a particular conjugate or composition. A suitable dosage for internal administration is 0.01 to 100 mg/kg per day. A preferred dosage is 0.01 to 35 mg/kg per day. A more preferred dosage is 0.05 to 5 mg/kg per day. A suitable concentration of the conjugate in pharmaceutical compositions for topical administration is 0.05 to 15% (by weight). A preferred concentration is from 0.02 to 5%. A more preferred concentration is from 0.1 to 3%. Ultimately, the attending physician will decide the dosage and the amount of conjugate of the present invention with which to treat each individual patient, taking into consideration a variety of factors, such as age, body weight, general health, diet, sex, conjugate or composition to be administered, route of administration, and severity of the disease being treated.

The conjugates of the present invention and the compositions thereof can be administered alone or in combination with other suitable components. Such components include those that aid in the delivery of a cytotoxic agent in a cell-specific manner or those that help the conjugates or compositions thereof treat cancer, for example.

One skilled in the art will appreciate that suitable methods of administering the conjugate of the present invention or composition thereof to an animal, e.g., a mammal such as a human, are known, and, although more than one route can be used to administer a particular composition, a particular route can provide a more immediate and more effective reaction than another route.

Formulations suitable for oral administration can consist of (a) liquid solutions, such as an effective amount of the conjugate dissolved in diluents, such as water or saline, (b) capsules, sachets or tablets, each containing a predetermined amount of the active ingredient, as solids or granules, (c) suspensions in an appropriate liquid, and (d) suitable emulsions.

Tablet forms can include one or more of lactose, mannitol, cornstarch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin or sucrose and acacia emulsions, gels, and the like containing, in addition to the active ingredient, such carriers as are known in the art.

Formulations suitable for parenteral administration include aqueous and non-aqueous solutions, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

### EXAMPLES

The following examples further illustrate the invention but, of course, should not be construed as in any way limiting its scope.

Abbreviations

For convenience, the following abbreviations are used herein: LHRH, leutinizing hormone releasing hormone; GRP, gastrin-releasing peptide; (SfY), sulfotyrosine; (OH), carboxylated peptide at either the N- or C-terminus; (Nle) norleucine; CCKA, cholecystokinin A; CCKB, cholecystokinin B; VIP-1, vasoactive intestinal peptide 1; NK-1, neurokinin 1; Dov, N,N-dimethylvaline; MeVal, N-methylvaline; Fmoc, 9-fluorenylmethoxycarbonyl; NMP, N-methylpyrrolidone; HBTU, 2-(1H-Benzotriazole-1-yl)-1,1,3,3-tetramethyluronium hexafluorophosphate; HOBt, N-Hydroxybenzotriazole; CIP, 2-chloro-1,3-dimethylimidazolidium; HOAt, 1-hydroxy-7-azabenzotriazole; DIPEA, diisopropylethylamine; DBU, 1,8-diazabicyclo[5.4.0]undec-7-ene; THF, tetrahydrofuran; HPLC, high performance liquid chromatography; LC/MS, liquid chromatography/mass spectrometry; ¹H-NMR, proton nuclear magnetic resonance; DCM, dichloromethane; MTT, methylthiazolyldiphenyl-tetrazolium bromide; PyBOP, (Benzotriazol-1-yloxy)tripyrrolidinophosphonium hexafluorophosphate; DIEA, N,N-diisopropylenthylamine; N-Boc, N-tert-butyl-oxycarbonyl; EtOAc, ethyl acetate; FEP, 2-fluoro-1-ethyl pyridinium tetrafluoroborate; TFA, trifluoroacetic acid; DMF, dimethylformamide; and ESI-MS, electro-spray ionization mass spectrometry.

### Example 1

This example demonstrates a method of synthesizing a conjugate of the present invention.

Solid Phase Peptide Synthesis of N,N-dimethylvaline (Dov)-Val-N-methylvaline (Me Val)-Pro-Pro-OH (Cemadotin derivative with free carboxy-terminus): Preloaded 9-fluorenylmethoxycarbonyl-proline-NovaSyn TGT (Fmoc-Pro-NovaSyn TGT) resin (0.80 g) was allowed to swell in N-methylpyrrolidone (NMP) for 2 hours prior to cleavage of the Fmoc protecting group. After deprotection with 20% piperidine in NMP, the second Pro residue was coupled via ABI 433 peptide synthesizer (Applied Biosystems, Foster City, Calif.) using 2-(1H-Benzotriazole-1-yl)-1,1,3,3-tetramethyluronium hexafluorophosphate (HBTU) and N-Hydroxybenzotriazole (HOBt) as coupling reagents. The remaining amino acid residues were coupled manually using 2-chloro-1,3-dimethylimidazolidium (CIP), 1-hydroxy-7azabenzotriazole (HOAt), and diisopropylethylamine (DIPEA) activation mixture (Akaji et al., Tetrahedron Letters 35: 3315-3318 (1994)). All the above reagents were purchased from Novabiochem (La Jolla, Calif.) except N,N-dimethyl-L-valine, which was prepared from L-valine by reductive alkylation with formaldehyde and NaCNBH₃ essentially as described in Ang et al., Aust. J. Chem. 44: 1591-1601 (1991).

Fmoc-MeVal-OH (0.33 g, 0.938 mmol) was dissolved in 6 ml of NMP. HOAt (0.06 g, 0.469 mmol) was added to the solution, followed by CIP (0.25 g, 0.906 mmol) and DIPEA (0.43 ml, 2.7 mmol). The activation mixture was added to the resin, and the residue was coupled overnight. The reaction mixture was drained and washed with NMP. Deprotection was achieved with 2% 1,8-diazabicyclo[5.4.0]undec-7-ene (DBU), 2% piperidine in NMP. Fmoc-Val-OH (1 mmol) was dissolved in 6 ml of NMP, followed by HOAt (0.10 g, 0.781 mmol), CIP (1.51 mmol) and DIPEA (0.65 ml, 3.75 mmol). The activation mixture was added to the washed resin, and the residue was double-coupled over 15 hours. Dimethylvaline (0.14 g, 0.938 mmol) was dissolved in 6 ml NMP, followed by the addition of HOAt (0.096 g, 0.703 mmol), CIP (0.39 g, 1.40 mmol), and DIPEA (0.59 ml, 3.38 mmol). The activation mixture was added to the deprotected resin, and the residue was coupled three times over 24 hours. Once all couplings were completed, the resin was washed with large quantities of NMP, CH₂Cl₂, and tetrahydrofuran (THF); the resin was then dried under vacuum overnight.

For cleavage of the peptide from the resin, a cleavage cocktail consisting of 100 μL of thioanisol, 50 μL water, 50 μL ethanedithiol, and 1.8 ml trifluoroacetic acid was used. The cocktail and the resin were chilled for 20 minutes, and then the mixture was added to the resin at 0° C. The resin slurry was stirred with a micro stir bar for 15 min at this temperature, and then the reaction was allowed to stir at room temperature for 1 hr 45 min. The resin was filtered using a disposable filtration column. Ether was used to precipitate the peptide. The resulting pentapeptide was purified by high performance liquid chromatography (HPLC) using a Zorbax C18 10×300 mm column (Agielent, Wilmington, Del.) in a gradient of 0.05% trifluoroacetic acid/water-acetonitrile. Structure and purity was confirmed by liquid chromatography/mass spectrometry (LC/MS) on Ion-Spray mass spectrometer (Agielent, Wilmington, Del.). Calculated mass: 552.4; found: 552.3.

Synthesis of cemadotin-peptide conjugates: Gastrin-linker merged sequences VLALAEEEAYGW(Nle)DF and FLALAEEEAYGW(Nle)DF were prepared by automated solid-phase peptide synthesis on ABI Rink amide resin (Applied Biosystems, Foster City, Calif.) utilizing ABI 433 peptide synthesizer (Applied Biosystems, Foster City, Calif.) equipped with conductivity monitoring system. Standard Fast Fmoc chemistry with HBTU/HOBt activation mixture was used (see Chan et al., Fmoc Solid Phase Peptide Synthesis—A Practical Approach, Oxford University, New York (2000)). Double-coupling was used for the last four N-terminal residues. The purity of the products was confirmed by analytical cleavage and LC/MS. Cemadotin with free carboxy terminus (27.6 mg, 0.05 mmol) in 0.5 ml NMP was activated by treatment with HOAt (3.2 mg, 0.025 mmol), CIP (12.4 mg, 0.045 mmol) and DIPEA (0.017 ml, 0.1 mmol). The mixture was added to 157 mg resin containing 0.274 mmol protected VLALAEEEAYGW(Nle)DF per 1 g (0.045 mmol) in 0.5 ml NMP. The mixture was incubated overnight, washed with NMP, dichloromethane (DCM) and dried. Cleavage from the resin and precipitation with ether was performed as described above. The product was purified by HPLC on C3 reverse phase column (10×300 mm) in the gradient of 0.05% trifluoroacetic acid/water-acetonitrile. Calculated molecular mass: 2260.3. Found by LC/MS-2260.2.

### Example 2

This example demonstrates the synthesis of conjugates comprising the hemiasterlin derivative, SPA110, and the gastrin decapeptide.

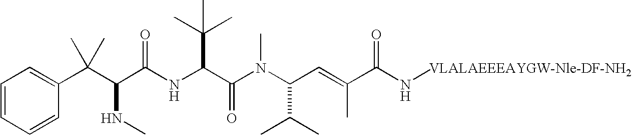

SPA 110 was prepared essentially as described previously (see R. Andersen et al. (WO99/32509)). SPA is a tripeptide that consists of three unnatural amino acids: (2E,4S)-2,5-dimethyl-4-(methylamino)-2-hexanoic acid, L-tert-leucine, and (2S)-N-methyl-3-methyl-3-phenylbutanoic acid. Due to significant steric difficulties, conjugation of all three was performed in solution rather than on solid phase. The resulting protected derivative of SPA 110 was attached to a peptide-ligand-linker sequence on a resin. Detailed description of the preparation of all intermediates are as follows.

### Preparation of N-(tert-Butoxycarbony)-N-methyl-L-valine-N′-methoxy-N′-methylamide (MD006)

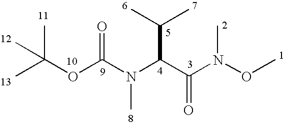

N,N-diisopropylethylamine (1.9 ml, 10.8 mmol) and (benzotriazol-1-yloxy)tripyrrolidinophosphonium hexafluorophosphate (PyBOP) (5.63 g, 10.8 mmol) were added to the cooled (0° C.), stirred solution of N-(tert-Butoxycarbony)N-methyl-L-valine (2.5 g; 10.8 mmol) in dichloromethane (18 ml). After 5 minutes, N,O-dimethylhydoxylamine hydrochloride (1.18 g (98% purity), 11.9 mmol) and N,N-diisopropylethylamine (2.1 ml, 11.9 mmol) were added to the mixture under an argon atmosphere. The solution was stirred at room temperature for 2.5 h. During the stirring, an additional portion of N,N-diisopropylenthylamine (DIEA) was added (1.2 ml total). The reaction mixture was diluted with dichloromethane (250 ml) and washed successively with aqueous 10% potassium hydrogen sulfate (3×30 ml), 10% aqueous sodium hydrogen carbonate (3×30 ml), and brine (3×30 ml). The organic phase was dried with magnesium sulfate and concentrated in vacuo to yield a pale yellow oil. The crude product was purified by column chromatography (4×60 cm, silica gel, 1:4 ethyl acetate-pentane), affording the product, MD0106, as clear colorless oil in 64% yield (1.884 g, 6.87 mmol).

Proton-nuclear magnetic resonance (¹H-NMR) (CDCl₃; 300 MHz), doubling of peaks caused by rotamers around the N-tert-butyl-oxycarbonyl (N-Boc) group, 5.00 (d, 0.55H, J=9.6 Hz, H-4), 4.71 (d, 0.45H, J=9.6 Hz, H-4), 3.73 (s, 1.8H, H-1), 3.69 (s, 1.25H, H-1), 3.21 (bs, 3H, H-2), 2.83 (s, 1.7H, H-8), 2.80 (s, 1.3H, H-8), 2.36-2.15 (m, 1H, H-5), 1.49 (s, 3.4H, H-11, H-12, H-13), 1.46 (s, 5.6H, H-11, H-12, H-13), 0.90 (d, ˜2H, J=6.0 Hz, H-6 and H-7), 0.88 (d, ˜4H, J=6.6 Hz, H-6 and H-7). TLC (ethyl acetate (EtOAc):Pentane 1:4) R_{f}0.54.

### Preparation of N-(tert-Butoxycarbony)-N-methyl-L-valinal (MD007)

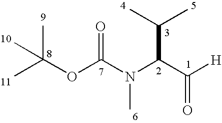

N-(tert-Butoxycarbony)-N-methyl-L-valine-N′-methoxy-N′-methylamide (1.884 g, 6.87 mmol) was dissolved under an argon atmosphere in THF (80 ml), and cooled to −78° C. Lithium aluminum hydride (330 mg (95%), 8.26 mmol) was then added. The reaction was stirred for 1.5 h, warmed to room temperature, and stirred for 30 min. The reaction mixture was then cooled and quenched with sodium sulfate decahydrate (6.87 mmol), and allowed to warm to room temperature. Celite was added to the solution and then it was filtered. Excess solvent was removed in vacuo to yield a colorless oil (1.260 g).

Crude material was purified by column chromatography (2.5×45 cm, silica gel, 1:6 ethyl acetate-hexanes) to give 61% yield (0.905 g, 3.30 mmol) of pure MD007.

¹H-NMR (CDCl₃, 400 MHz), doubling of peaks caused by rotamers around the N-Boc group, 9.66 (bs, 1H, H-1), 4.09 (d, 0.5H, J=9.6 Hz, H-2), 3.63 (d, 0.5H, J=8.8 Hz, H-2), 2.91 (s, 1.5H, H-6), 2.81 (s, 1.5H, H-6), 2.27 (bm, 1H, H-3), 1.47 (s, 4.5H, H-9, H-10, H-11), 1.45 (s, 4.5H, H-9, H-10, H-11), 1.10 (d, 3H, J=6.4 Hz, H-4 and H-5), 0.93 (d, 3H, J=6.8 Hz, H-4 and H-5). TLC (EtOAc:Hexane 1:6) R_{f}=0.48.

### Preparation of (2E,4S)-2,5-dimethyl-4-(methylamino)-2-hexanoic Acid Ethyl Ester (MD008)

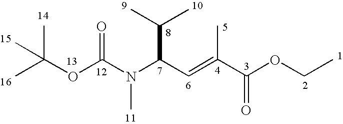

(Carbethoxyethylidene)triphenylphosphane (1.783 g (94%), 4.62 mmol) was added to the aldehyde of MD007 (905 mg, 4.20 mmol), which was dissolved in methylene chloride (50 ml). The reaction was stirred at room temperature under an argon atmosphere for 3.5 h and then overnight (20 h) at 50° C. The solvent was evaporated and the residue was purified by column chromatography (2.5×45 cm, silica gel, 1:6 ethyl acetate-hexanes) to give a 54% product yield (682 mg, 2.28 mmol).

¹H-NMR (CDCl₃, 400 MHz), doubling of peaks caused by rotamers around the N-Boc group, 6.66 (d, 1H, J=8.8 Hz, H-6), 4.57 (bs, 0.5H, H-7), 4.31 (bs, 0.5H, H-7),4.21 (q, 2H, J=7.1 Hz, H-2), 2.72 (bs, 3H, H-11), 1.91 (s, 3H, H-5), 1.87 (bs, 1H, H-8), 1.46 (s, 9H, H-14, H-15, H-16), 1.31 (t, 3H, J=7.2 Hz, H-1), 0.91 (d, 3H, J=6.4 Hz, H-9 and H-10), 0.86 (d, 3H, J=6.4 Hz, H-9 and H-10).). TLC (EtOAc:Hexane 1:6) R_{f}0.47.

### Preparation of N-(tert-Butoxycarbony)-L-tert-leucine (MD009)

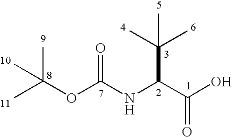

L-tert-Leucine (2.63 g, 20 mmol) was dissolved in a mixture of water (50 ml), 1,2-dioxan (20 ml), and 0.8 g sodium hydroxide, cooled in an ice bath. Di-tert-Butoxycarbonate (4.8 g, 22 mmol) in 1,2-dioxan (20 ml) was added dropwise to the cooled solution. The reaction mixture was stirred at room temperature for 4 h and the pH was adjusted between 8 and 9. Dioxane was then evaporated in vacuo and the resulting solution was acidified with 10% aqueous potassium hydrogen sulfate to pH 3 and extracted with ethyl acetate (4×50 ml). The combined organic layers were washed with 10% aqueous potassium hydrogen sulfate (3×30 ml), brine (3×30 ml), and water (3×30 ml). The organic phase was dried with anhydrous magnesium sulfate and concentrated in vacuo to give a white solid. This resulted in a 89% yield (4.133 g, 17.87 mmol).

¹H-NMR (CDCl₃, 400 MHz), doubling of peaks caused by rotamers around the N-Boc group, 5.81 (bs, 0.2H, NH), 5.09 (d, 0.8H, J=8.8 Hz, NH), 4.13 (d, 0.8H, J=9.6 Hz, H-2), 3.91 (bs, 0.2H, H-2), 1.45 (s, 9H, H-9, H-10, H-11), 1.02 (s, 9H, H-4, H-5, H-6). TLC (MeOH:CH₂Cl₂ 5:20) R_{f}=0.91, (MeOH:H₂O:CH₂Cl₂ 45:5:200) R_{f}0.86.

### Preparation of Boc-Tle-N-Me-Val-OEt (MD010)

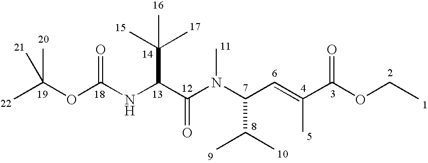

HOAt (35 mg, 2 eq.), CIP (142.3 mg, 2 eq.) and DIEA (0.266 ml, 6 eq.) were added to the solution of N-(tert-Butoxycarbony)-L-tert-leucine (118 mg, 0.51 mmol, 2 eq.) in 2 ml methylene chloride. After one minute, this solution was added to the solution of MD009 (73 mg, 0.244 mmol, 1 eq.) in 2 ml methylene chloride. The reaction mixture was stirred under an argon atmosphere for 4 h at room temperature. The solvent was removed in vacuo, and the residue was extracted with 70 ml ethyl acetate. The organic layer was washed with 10% aqueous potassium hydrogen sulfate (3×30 ml), 10% aqueous sodium hydrogen carbonate (3×30 ml), brine (3×30 ml), and then dried with anhydrous magnesium sulfate. After concentration, the crude product was purified by column chromatography (1×40 cm, silica gel, ethyl acetate-hexane 1:6) to give a 45% yield (45 mg, 0.109 mmol).

¹H-NMR (CDCl₃, 500 MHz), doubling of peaks caused by rotamers around the N-Boc group, 6.65 (dq, 1H, J₁=9.5 Hz, J₂=1.5 Hz, H-6), 5.23 (bd, 1H, J=5.0 Hz, NH), 5.12 (t, 1H, J=9.7 Hz, H-7), 4.43 (d, 1H, J=10.5 Hz, H-13), 4.21 (q, 2H, J=7.0 Hz, H-2), 2.99 (s, 3H, H-11), 1.94-1.85 (m, 4H, H-5 and H-8), 1.42 (s, 9H, H-20, H-21 and H-22), 1.31 (t, 3H, J=7.0 Hz, H-1), 1.01 (s, 1.2H, H-15, H-16 and H-17), 0.96 (s, 7.8H, H-15, H-16 and H-17), 0.88 (d, 3H, J=6.5 Hz, H-9 or H-10), 0.84 (d, 3H, J=6.5 Hz, H-1 or H-9).

TLC (EtOAc:hexane 1:6) R_{f}0.35.

### Preparation of 3-Methyl-3-phenylbutanoic Acid (MD015)

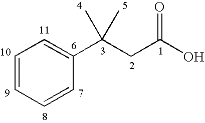

3-methyl-2-butenoic acid (97%, 5.26 g, 51.0 mmol) and AlCl₃ (20.4 g, 153 mmol) were placed in a one-neck round-bottomed flask (250 ml) under an argon atmosphere. Benzene (50 ml, 560 mmol) was added producing vigorous bubbling. Upon completion of the bubbling, a condenser capped by a balloon with argon was attached. The reaction mixture was stirred in an oil bath at 65° C. for 1 h and 35 min. Diethyl ether was added to the solution and the mixture was cooled to 0° C. Concentrated HCl and water were added slowly until the entire solid dissolved and the pH was less than 2. The aqueous layer was extracted with diethyl ether four times. The organic layer was concentrated to 150 ml and extracted with a saturated sodium hydrogen carbonate solution six times (6×150 ml). The combined aqueous layers were acidified with concentrated HCl until the pH was less than 2. The acidic aqueous layer was extracted with diethyl ether four times and the accumulated organic layer was dried with magnesium sulfate. The solution was filtered and the diethyl ether was removed in vacuo, producing a white solid (4.42 g, 24.8 mmol) in 46% yield, which did not need further purification, mp 55.5-57.0° C.

¹H-NMR (400 MHz, CDCl₃) 11.25 (bs, 1H, CO₂H), 7.37 (d, 2H, J=7.6 Hz, H-11 and H-7), 7.31 (t, 2H, J=7.2 Hz, H-10 and H-8), 7.20 (t, 1H, J=7.2 Hz, H-9), 2.65 (s, 2H, H-2), 1.47 (s, 6H, H-4 and H-5).

### Preparation of (4S)-3-(3-methyl-3-phenyl-1-oxobutyl)-4-isopropyl-2-oxazolidinone (MD017B)

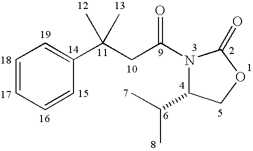

3-Methyl-3-phenylbutanoic acid (MD015, 2.48 g, 13.91 mmol) was dissolved in 170 ml of THF and cooled to −78° C. Triethylamine (2.90 ml, 20.80 mmol) and trimethylacetyl chloride (1.88 ml, 15.11 mmol) were added to the reaction flask producing a white solid. The resulting mixture was warmed to 0° C. for 1 h and 10 min and then cooled back down to −78° C. In a second flask, butyllithium (10.65 ml, 2.5 M in hexanes, 25.63 mmol) was added dropwise with vigorous stirring to a solution of (4S)-(−)-4-isopropyl-2-oxazolidinone (3.55 g, 27.45 mmol) at −78° C. in THF (120 ml), producing a white precipitate. The resulting suspension of the lithiated oxazolidinone was added via cannula to the reaction flask. Stirring was continued for 2 h and 20 min. Water was added and the reaction mixture was warmed to room temperature, whereupon it was extracted four times with diethyl ether. The combined organic extracts were dried over magnesium sulfate, and concentrated in vacuo. The product was purified by flash column chromatography (silica gel, 4.5×60 cm, diethyl ether-hexanes 3:7), affording compound MD017B as a clear, colorless oil in 91.6% yield (3.69 g, 12.61 mmol).

¹H-NMR (400 MHz, CDCl₃) 7.40 (d, 2H, J=7.2 Hz, H-19 and H-15) 7.30 (t, 2H, J=7.2 Hz, H-18 and H-16), 7.18 (t, 1H, J=7.2 Hz, H-17), 4.24-4.20 (m, 1H, H-4), 4.08 (dd, 1H, J=9.2 and 2.8 Hz, 1H-5), 4.02 (t, 1H, J=9.2 Hz, 1H-5), 3.40-3.31 (m, 2H, H-10), 2.22-2.10 (m, 1H, H-6), 1.50 (s, 3H, H-13 or H-12), 1.49 (s, 3H, H-13 or H-12), 0.81 (d, 3H, J=6.80 Hz, H-8 or H-7), 0.74 (d, 3H, J=6.80 Hz, H-8 or H-7). TLC (diethyl ether:Hexane 3:7) R_{f}=0.31.

### Preparation of 4-isopropyl-2-oxazolidinone (MD019)

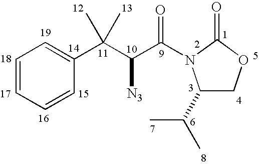

Oxazolidinone MD017B (3.672 mg, 12.71 mmol) was dried under high vacuum for 1 h, dissolved in THF (78 ml) under an argon atmosphere, and cooled to −78° C. Freshly prepared solution of potassium bis(trimethylsilyl)amide (120.0 ml, 0.1166 M in THF, 13.99 mmol) was added and the resulting solution was stirred at −78° C. for 1 h and 20 min. A solution of 2,4,6-triisopropylbenzenesulfonyl azide (4.870 g, 15.74 mmol) in THF (39 ml) at −78° C. was added via cannula and after 4.5 min, the orange colored reaction mixture was treated with glacial acetic acid (3.35 ml, 58.52 mmol), warmed to 40° C. in a water bath (13 min.), and stirred for an additional 1 h 20 min. Brine (270 ml) and water (35 ml) were added to the light yellow mixture and the aqueous phase was extracted five times with 500 ml diethyl ether. The combined organic extracts were washed with a saturated sodium hydrogen carbonate solution (2×1 10 ml), dried with magnesium sulfate, and concentrated in vacuo. The product was purified by column chromatography (5×55 cm, silica gel, 3:7 diethyl ether-hexanes, sample was loaded with diethyl ether), affording azide MD019 as a colorless oil (3.64 g, 11.02 mmol) in 86.8% yield.

¹H-NMR (400 MHz, CDCl₃) 7.41 (d, 2H, J=7.2 Hz, H-19 and H-15), 7.32 (t, 2H, J=7.2 Hz, H-18 and H-16), 7.25 (t, 1H, J=7.2 Hz, H-17), 5.66 (s, 1H, H-10), 3.97 (dd, 1H, J=9.0 and 2.2 Hz, 1H-5), 3.91-3.87 (m, 1H, H-4), 3.58 (t, 1H, J=8.4 Hz, 1H-5), 2.37-2.25 (m, 1H, H-6), 1.56 (s, 3H, H-13 or H-12), 1.54 (s, 3H, H-13 or H-12), 0.85 (d, 3H, J=6.8 Hz, H-8 or H-7), 0.81 (d, 3H, J=7.2 Hz, H-8 or H-7); optical rotation obtained was +113.02 (c 1.392 CHCl₃). TLC (diethyl ether:hexane 3:7) R_{f}0.52.

### Preparation of 4-isopropyl-2-oxazolidinone (MD020)

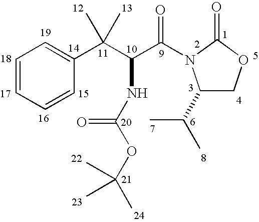

Azide MD019 (1.300 g, 3.93 mmol) was dissolved in 120 ml of ethyl acetate and flushed with argon, then di-tert-butyl dicarbonate (1.926 g, 8.82 mmol) and 10% palladium on charcoal (910 mg) were added and the resulting black suspension was stirred at room temperature. The mixture was flushed with argon, then with hydrogen (5.5 h) and was stirred under a hydrogen balloon overnight (15 h). Finally, it was all once again flushed with hydrogen for 1.5 h.

The reaction mixture was flushed with argon and was filtered through silica gel and the collected material was washed with ethyl acetate. The combined filtrate was concentrated in vacuo and the crude mixture was purified by flash column chromatography (2.5×46 cm, silica gel, 3:7 diethyl ether-hexanes) to afford compound MD020 as a viscous colorless oil, in 86% yield (1.371 g, 3.39 mmol).

¹H-NMR (400 MHz, CDCl₃) 7.42 (d, 2H, J=7.6 Hz, H-19 and H-15), 7.32 (t, 2H, J=7.6 Hz, H-18 and H-16), 7.24 (t, 1H, J=7.6 Hz, H-17), 6.14 (d, 1H, J=9.6 Hz, H-10), 5.14 (bs, 1H, N—H), 3.89 (dd, 1H, J=8.8 and 2.0 Hz, H-5), 3.85-3.80 (m, 1H, H-4), 3.47 (t, 1H, J=8.4 Hz, H-5), 2.31-2.20 (m, 1H, H-6), 1.49 (s, 9H, H-24, H-23 and H-22), 0.83 (d, 3H, J=7.2 Hz, H-8 or H-7), 0.78 (d, 3H, J=6.8 Hz, H-8 or H-7); optical rotation obtained was +118.33 (c 1.904 CHCl₃). TLC (diethyl ether:hexane 3:7) R_{f}=0.33.

### Preparation of Methyl (2S)-2-(tert-butyloxycarbonyl)amino-3-methyl-3-phenylbutanoate (MD021) and (2S)-2-(tert-butyloxycarbonyl)amino-3-methyl-3-phenylbutanoic Acid (MD021a)

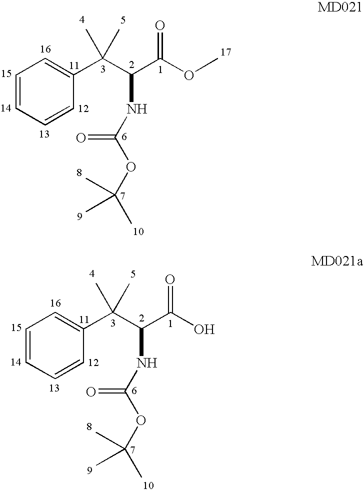

Oxazolidinone MD020 (1.360 g, 3.36 mmol) was dissolved in a mixture of 40 ml THF and 10 ml water. The solution was cooled to 0° C. Hydrogen peroxide (3.44 ml, 30% aqueous, 33.68 mmol) and lithium hydroxide (10.12 ml, 1.0 M, 10.12 mmol) were then added to the oxazolidinone solution and stirred at room temperature overnight (20 h). The excess peroxide was quenched by addition of sodium hydrogen sulfite (40 ml, 1.5 M, 60 mmol) and stirring was continued for 1 h. The aqueous phase was acidified with 1.0 M citric acid and the mixture was extracted with ethyl acetate (2×200 ml, 2×150 ml, 1×100 ml). The combined ethyl acetate extracts were dried over magnesium sulfate and concentrated in vacuo. Solution of diazomethane in diethyl ether was added to the remaining crude material until the solution stayed yellow. After bubbling argon through the solution for 15 min, the remaining volatile components were removed in vacuo to afford the crude compound MD021. Purification of ester MD021 was accomplished by column chromatography (3.5×45 cm, silica gel, 3:7 diethyl ether-hexanes; sample was loaded with CHCl₃), producing two fractions: MD020, a clear colorless oil (337 mg, 1.10 mmol, 33%), and the acid MD021a, a colorless oil (467 mg, 1.59 mmol, 47% yield). The overall yield was 80%.

MD021, ¹H-NMR (400 MHz, CDCl₃) 7.37-7.29 (m, 4H, H-16, H-15, H-13, H-12), 7.22 (t, 1H, J=6.6 Hz, H-14), 5.02 (bm, 1H, H-2), 4.52 (bd, 1H, J=9.2 Hz, N—H), 3.51 (s, 3H, H-17), 1.43 (s, 3H, H-5 or H-4), 1.40 (s, 3H, H-5 or H-4), 1.39 (s, 9H, H—I 0, H-9, and H-8). TLC (diethyl ether:hexane 3:7) R_{f}=0.58.

MD021a, ¹H-NMR (200 MHz, CDCl₃) 7.40-7.20 (m, 5H, H-16, H-15, H-13, H-12, H-14), 4.94 (bd, 1H, J=8.8 Hz, H-2), 4.55 (bd, 1H, J=8.8 Hz, N—H), 1.45 (s, 3H, H-5 or H-4), 1.46 (s, 3H, H-5 or H-4), 1.38 (s, 9H, H-10, H-9, and H-8).

### Preparation of (2S)-N-tert-butoxycarbonyl-N-methyl-3-methyl-3-phenylbutanoic Acid (MD022)

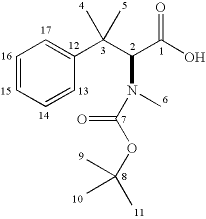

Procedure A

Sodium hydride (250 mg, 10.4 mmol) and a catalytic amount of tetrabutylamonium iodide were added under an argon atmosphere to a vigorously stirred solution of ester MD021 (115.0 mg, 0.374 mmol) in 5 ml dry dimethylformamide (DMF) followed by methyl iodide (0.230 ml, 3.69 mmol). The resulting gray suspension was stirred overnight (20.5 h) at room temperature. The excess sodium hydride was quenched by cautious addition of water (ice bath) and the mixture was acidified by dropwise addition of 1.0 M citric acid. The acidic mixture was extracted four times with ethyl acetate; the combined organic layer was extracted three times with brine, dried over magnesium sulfate, and then concentrated in vacuo. The resulting light orange oil was dissolved in 8 ml methanol in a 50 ml flask. Water (2.0 ml) was added to the solution, followed by 2.9 ml of 1.0 M lithium hydroxide. The reaction mixture was heated under an argon atmosphere at 60-65° C. overnight (17 h), producing a white precipitate. The aqueous layer was acidified with 1.0 M citric acid to pH 3. The mixture was extracted four times with ethyl acetate. The combined organic layers were dried with magnesium sulfate and concentrated in vacuo to give 165 mg of crude material. Purification of acid MD022 was performed by silica gel column chromatography (2×40 cm, 1:2 diethyl ether-hexanes with 1% acetic acid), resulting in a 97% yield (115.0 mg, 0.374 mmol) of a clear colorless oil.

¹H-NMR (400 MHz, CDCl₃) 7.43 (d, 1.3H, J=8.0 Hz, H-17 and H-13), 7.39 (d, 1.3H, J=8.0 Hz, H-17 and H-13), 7.30 (t, 2H, J=7.6 Hz, H-16 and H-14), 7.20 (t, 1H, J=7.4 Hz, H-15), 5.11 (s, 0.66H, H-2), 4.95 (s, 0.33H, H-2), 2.77 (s, 1.H, H-6), 2.63 (s, 2H, H-6), 1.56 (s, 3H, H-5 or H-4), 1.51-1.39 (m, 12H, H-5 or H-4 and H-11, H-10 and H-9). TLC (diethyl ether:hexane 1:2+1% AcOH)R_{f}=0.40.

### Preparation of (2S)-N-tert-butoxycarbonyl-N-methyl-3-methyl-3-phenylbutanoic Acid (MD022)

Procedure B

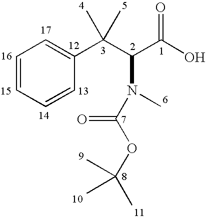

Under an argon atmosphere, sodium hydride (1.20 g, 48 mmol), a catalytic amount of tetrabutylamonium iodide, followed by methyl iodide (2.0 ml, 32 mmol) was added to a vigorously stirred solution of acid MD021a (467 mg, 1.59 mmol) in 20 ml dry DMF. The resulting grey suspension was stirred overnight (20.5 h) at room temperature. The excess sodium hydride was quenched by cautious addition of water (ice bath) and the mixture was acidified by dropwise addition of 1.0 M citric acid to pH 3. The acidic mixture was extracted four times with ethyl acetate; the combined organic layer extracted three times with brine, dried over magnesium sulfate and concentrated in vacuo. The resulting light orange oil was dissolved in 40 ml methanol in a 250 ml flask. Water (10.0 ml) was added to the solution, followed by 12.2 ml of 1.0 M lithium hydroxide. The reaction mixture was heated under argon at 60-65° C. overnight (17 h), producing a white precipitate. The aqueous layer was acidified with 1.0 M citric acid to pH 3. The mixture was extracted four times with ethyl acetate. The combined organic layers were dried with magnesium sulfate and concentrated in vacuo to give a 1.732 g of crude material. Purification of acid MD022 was performed by silica gel column chromatography (5×55 cm, 1:2 diethyl ether-hexanes with 1% acetic acid) resulting in a 77% yield (374.2 mg, 1.217 mmol) of a clear colorless oil.

¹H-NMR (400 MHz, CDCl₃) 7.43 (d, 1.3H, J=7.6 Hz, H-17 and H-13), 7.39 (d, 1.3H, J=8.00 Hz, H-17 and H-13), 7.32 (t, 2H, J=7.6 Hz, H-16 and H-14), 7.20 (t, 1H, J=7.2 Hz, H-15), 5.15 (s, 0.66H, H-2), 4.95 (s, 0.33H, H-2), 2.77 (s, 1.H, H-6), 2.63 (s, 2H, H-6), 1.57 (s, 3H, H-5 or H-4), 1.52-1.39 (m, 12H, H-5 or H-4 and H-11, H-10 and H-9). TLC (diethyl ether:hexane 1:2+1% AcOH)R_{f}=0.40.

### Preparation of MD023

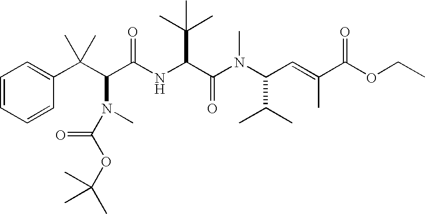

N-Boc-amino ester MD010IV (45 mg, 0.109 mmol) was dissolved in 1 ml CH₂Cl₂ under an argon atmosphere and 1 ml of trifluroracetic acid (TFA) was added. The reaction mixture was stirred at room temperature for 0.5 h. After removal of the solvent in vacuo, repeated rinsing of the remaining material with CH₂Cl₂ (3×5 ml) and evaporation of the residual solvent TFA salt of the amino acid ester, MD010IV was obtained.

N-Boc protected amino acid MD022 (36.9 mg, 0.12 mmol), HOAt (16.4 mg, 0.12 mmol), 2-fluoro-1-ethyl pyridinium tetrafluoroborate (FEP) (25.6 mg, 0.12 mmol) and DIEA (0.100 ml, 057 mmol) were added to the cooled solution (−10° C.) of the amino acid MD010IV TFA salt in 2.0 ml CH₂Cl₂ under argon. The solution was stirred for a few minutes at −10° C. and then for 2 h at room temperature.

Ethyl acetate (50 ml) was added to the mixture and the organic phase was washed with 10% aqueous sodium hydrogen carbonate (3×10 ml), brine (1×10 ml), 110/o aqueous potassium hydrogen sulfate (3×10 ml) and brine (3×10 ml). The organic layer was dried with magnesium sulfate and the solvent was removed in vacuo. The product was purified by column chromatography (silica gel, 1:1 diethyl ether-hexanes), affording the protected tripeptide MD023 as a clear colorless oil in 73% yield (0.0477 g, 0.0793 mmol).

Electro-spray ionization-mass spectrometry (ESI-MS) [M+H]⁺602.4/602.4 (expected/found). See Peng et al., Tetrahedron 56: 8119-8131 (2000), for a general protocol for amino acid coupling with FEP.

### Preparation of MD024

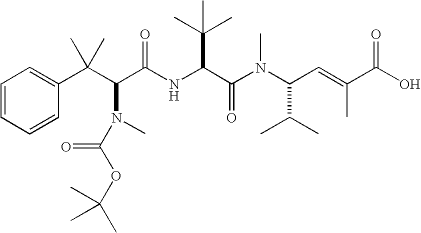

Water (0.62 ml) and 0.65 ml of a 1.0 M aqueous solution of lithium hydroxide (0.65 mmol) were added to a solution of the ethyl ester MD023 (47.7 mg, 0.0793 mmol) in 2.28 ml MeOH under argon. The reaction mixture was stirred at room temperature overnight (23 h), and then for 5 h at 32° C., whereupon it was acidified by dropwise addition of 1.0 M citric acid and then extracted four times with ethyl acetate. The combined organic extracts were dried with magnesium sulfate and concentrated in vacuo. A crude product was purified by short column filtration (silica gel, chloroform with 1% acetic acid) and after that by HPLC (ZORBAX 300SB-C39.4 mm×25 cm, water-acetonitryl (0.1% TFA), gradient mode from 0% ACN to 100% ACN in 90 min., flow 4 ml/min.). ESI-MS [M+H]⁺570.4/574.3 (expected/found).

### Preparation of MD025

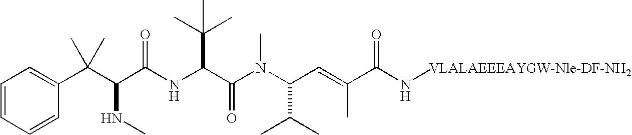

The amide resin (367 mg) with attached peptide Fmoc-VLALA-10G (0.278 mmol/g, 0.100 mmol) was deprotected with 25% piperidine in NMP (15 ml, 1×3 min and 3×7 min) and washed with NMP (6×20 ml for 1 min). The amino acid MD024 (29.0 mg, 0.050 mmol, 0.5 eq.), HOAt (110.6 μl 0.5 M solution, 0.055 mmol, 0.55 eq.), FEP-1-ethyl-2-fluoropyrydinium tetrafluoroborate (11.8 mg, 0.055 mmol, 0.55 eq.) in 4 ml NMP was added to the resin, then DIEA (28.9 μl, 0.174 mmol, 1.65 eq.) was added and the resin mixture was stirred under argon for 4 h. The resin was then washed with NMP (6×20 ml for 1 min) and the unreacted amino groups were capped using acetic anhydride/DIEA (3 eq./9 eq.) procedure. The resin was washed carefully with NMP (6×20 ml for 1 min), DCM (6×20 ml for 1 min), MeOH (6×20 ml for 1 min) and dried in vacuo overnight.

Resin was cooled to the −15° C. and cleaved by addition of cold mixture of: thioanisole, water, ethanodithiol, TFA (2:1:1:36, 2 ml). The cleavage mixture was stirred under argon for 15 min in −15° C., and then warmed to room temperature and stirred for additional 2 h. The resin was filtered, washed with TFA and the solution was precipitated with cold diethyl ether. The precipitate was washed four times with cold ether and dried in vacuo overnight.

Crude peptide was purified by reverse phase-high performance liquid chromatography (RP-HPLC) using ZORBAX 300SB-C3 9.4 mm×25 cm column (water-acetonitril (0.1% TFA) solvent system, gradient mode from 0% ACN to 100% ACN in 90 min, flow 4 ml/min.).

ESI-MS [M+H]⁺2180.2/2180.1, [M+2H]²⁺1090.6/1091.2, [M+3H]³⁺727.4/727.7 (expected/found).

### Example 3

This example describes the activity of the conjugate comprising the hemiasterlin derivative, SPA110, the linker VLALA, and the gastrin decapeptide.

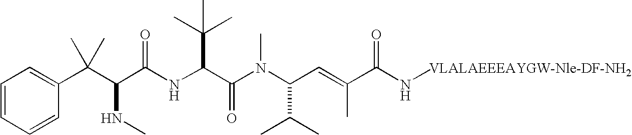

This conjugate had relatively low activity (IC₅₀=1 μM) when tested on gastrin receptor-expressing 3T3 cells in accordance with the methods of Example 7. The low activity observed appears to be due to insufficient processing of the conjugate in the lysosomes. Hydrolysis of the conjugate with two major lysosomal proteases, namely cathepsin B and cathespin D, generated mostly pentapeptide or SPA110 extended by Val-Leu on the C-terminus. Toxicity testing of synthesized SPA110 extended by Val-Leu on the C-terminus confirmed the observed results. Further enzymatic processing of HTI conjugates does not occur because proteases are not able to cleave after the β amino acid of HTI-286.

### Example 4

This example describes the generation of a library of HTI-286 derivatives extended by one α-amino acid at the C-terminus and their activity.

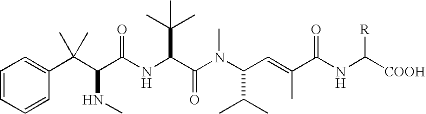

Resin containing 0.1 mmol of suitable Fmoc-protected amino acid was swelled for 1 hr in NMP, and then washed twice by the same solvent. The Fmoc protection group was removed with 25% piperidine/NMP solution (1×5′, 1×10′, 1×15′, 5 ml). The resin was washed five times with NMP and two times with anhydrous NMP and mixed with Boc-protected HTI-286 hemiasterlin derivative (0.025 mmol), FEP (1-ethyl-2-fluoropyrydinium tetrafluoroborate) (0.03 mmol), HOAt (1-hydroxy-7-azabenzotriazole; 3H-[1,2,3]-triazolo[4,5-b]pyridin-3-ol) (0.03 mmol) and DIEA (N,N-diisopropylethylamine) (0.125 mmol) in 4 ml of dry NMP. The reaction mixture was stirred by argon for 4 hr, and washed 5 times with NMP, DCM and finally MeOH. Traces of solvents were removed over vacuo.

The resin was cooled to −15° C. and cleaved by addition of a cold mixture of TFA (trfluoroacetic acid), water, and TIS (triisopropylsilane) (95:2.5:2.5, 3 ml). The cleavage mixture was stirred under argon atmosphere for 10 min in −15° C. and then 10 min in 0° C., and finally was warmed to room temperature and stirred for additional 80 min. The resin was filtered and washed with TFA, and the solution was evaporated on vacuo.

The crude peptide was purified by RP-HPLC using ZORBAX 300SB-C18 9.4 mm×25 cm column (water-acetonitryl (0.1% TFA)) solvent system, gradient mode from 0% ACN to 100% ACN in 50 min, flow 4 ml/min.).

The purity of the compounds was evaluated by HPLC-MS.

Upon testing on gastrin receptor-expressing 3T3 cells in accordance with the methods of Example 7, it was found that even slight variations in the structure of R had significant effects on activity. Two unnatural amino acids, namely 1-naphyhyl-alanine and cyclohexyl alanine (IC₅₀=1 nM), were found to be optimal. Charged residues, like Asp and Lys, on the other hand, produced totally inactive compounds. Substitution of the cyclohexyl group on alanine with a phenyl group reduced activity almost ten-fold.

### Example 5

This example describes the generation of a library of peptides of general formula 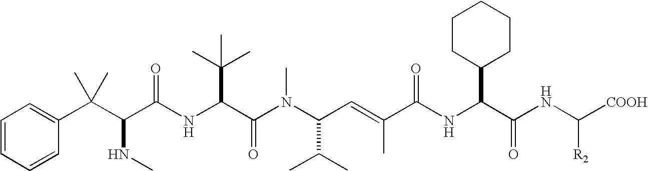
and their activity.

The dipeptide library of general structure 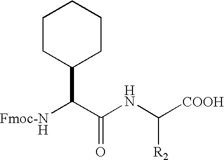
was prepared using preloaded Wang resins with suitable Fmoc-protected amino acid (0.1 mmol) on an ABI 433 peptide synthesizer (Applied Biosystems, Froster City, Calif.). After Fmoc deprotection using 20% piperidine in NMP, 9-fluorenylmethoxycarbonyl-cyclohexylalanine was coupled using HBTU (2-(1H-benzotriazole-1-yl)-1,1,3,3-tetramethyluronium hexafluorophosphonate) and HOBt (N-hydroxybenzotriazole) as coupling reagents.

Each resin from the dipeptide library was swelled for 1 hr in NMP and washed twice with the same solvent. Fmoc protection was removed with 25% piperidine/NMP solution (1×5′, 1×10′, 1×15′, 5 ml). The resin was washed five times with NMP and two times with anhydrous NMP and mixed with Boc-protected HTI-286 hemiasterlin derivative (0.025 mmol), FEP (1-ethyl-2-fluoropyrydinium tetrafluoroborate) (0.03 mmol), HOAt (1-hydroxy-7-azabenzotriazole; 3H-[1,2,3]-triazolo[4,5-b]pyridin-3-ol) (0.03 mmol) and DIEA (N,N-diisopropylethylamine) (0.125 mmol) in 4 ml of dry NMP. The reaction mixture was mixed by argon for 4 hr, and washed 5 times by NMP, DCM and finally MeOH. Traces of solvents were removed over vacuo.

The resin was cooled to −15° C. and cleaved by the addition of a cold mixture of TFA (trifluoroacetic acid), water, and TIS (triisopropylsilane) (95:2.5:2.5, 3 ml). The cleavage mixture was stirred under argon atmosphere for 10 min at −15° C., then 10 min at 0° C., finally was warmed to room temperature and stirred for an additional 80 min. The resin was filtered and washed with TFA and the solution was evaporated on vacuo.

The crude peptide was purified by RP-HPLC using ZORBAX 300SB-C18 9.4 mm×25 cm column (water-acetonitryl (0.1% TFA)) solvent system, gradient mode from 0% ACN to 100% ACN in 50 min, flow 4 ml/min.

The purity of the compounds was evaluated by HPLC-MS.

Upon testing on gastrin receptor-expressing 3T3 cells in accordance with the methods of Example 7, it was found that the most active compound of the series was the one in which R2=Cyclohexyl (IC₅₀=30 nM). The second most active compound of the series was the one in which R2=Leu (IC₅₀=120 nM). The gastrin conjugates potently and selectively inhibited the growth of gastrin receptor-expressing 3T3 cells. HTI-286-Cha-Leu-Ala-Leu-Ala-EEEAYGW-Nle-DF-NH₂ had an IC₅₀=10 nM (IC₅₀=300 nM on nontransfected cells, which express a low-affinity gastrin receptor).

### Example 6

This example describes a variety of cytotoxic agents that can be used in the conjugates of the present invention and illustrates the point at which the ligand-linker fused sequence is attached to the cytotoxic agents.

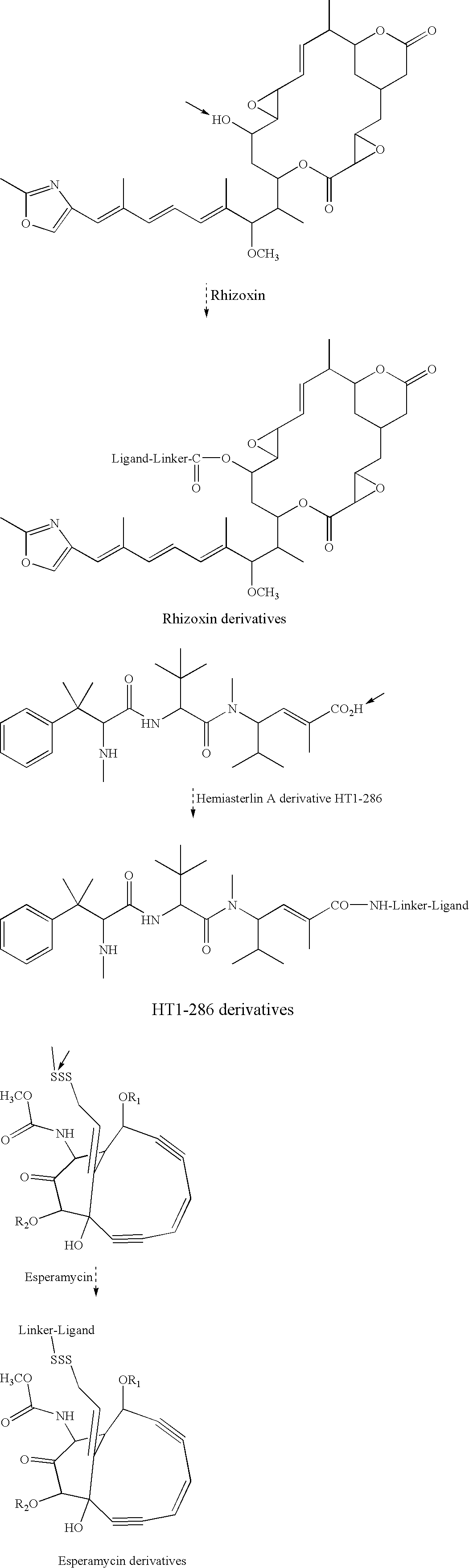

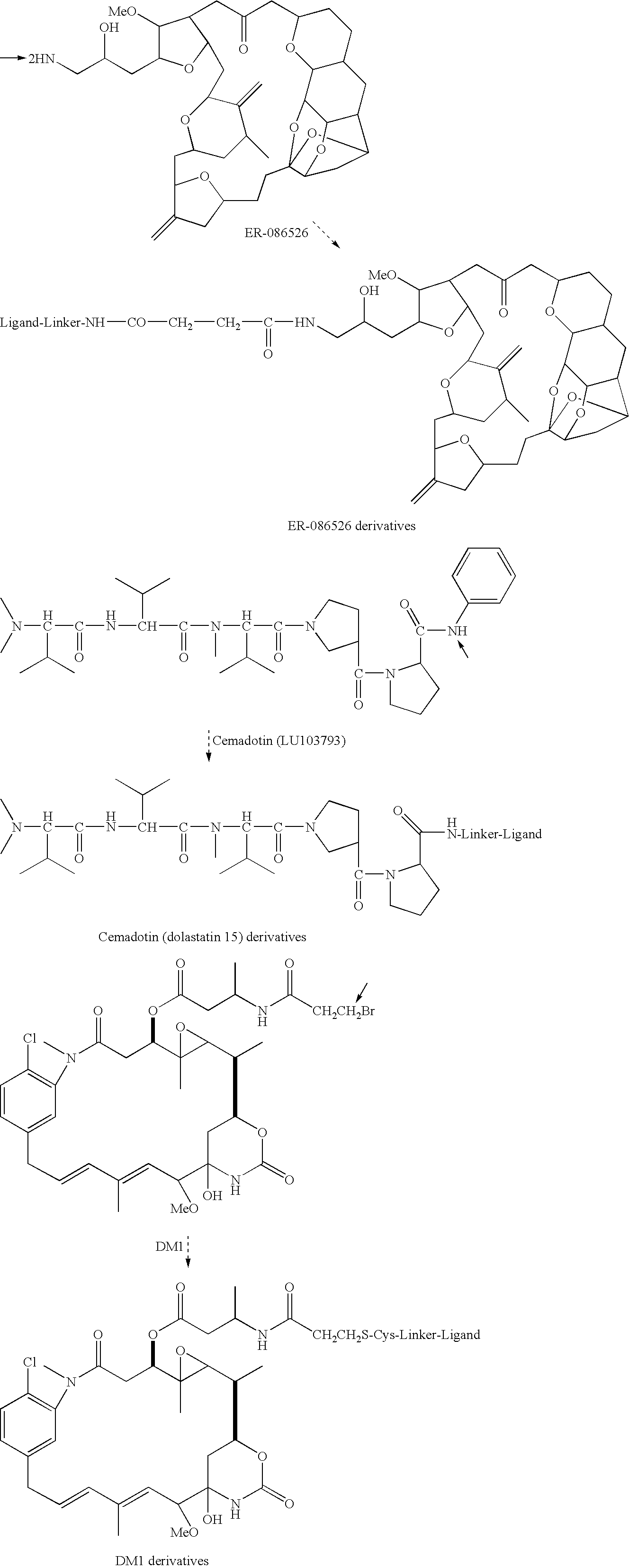

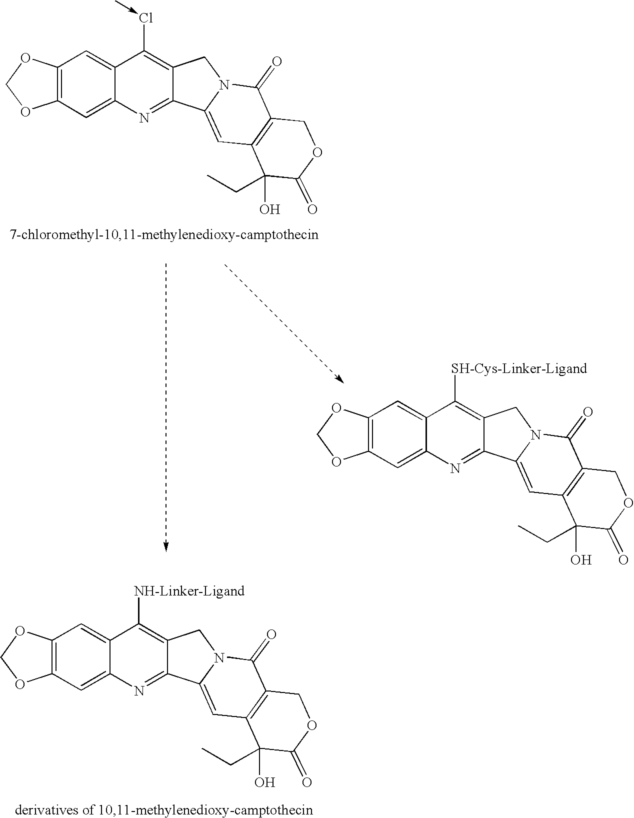

### Example 7

This example demonstrates an assay to test the cytotoxicity of the conjugates in vitro and demonstrates that administration of a conjugate, which comprises a ligand that specifically binds the gastrin receptor, to cells expressing the receptor leads to a dose-dependent decrease in cell survival.

Isogenic cell lines, one transfected with the target cell surface receptor and the parent cell line without detectable receptor expression, were used for determination of selective activity of toxin conjugates. Transfection of NIH/3T3, CHO and HeLa cells (American Type Culture Collection, Manassas, Va.) with gastrin receptor cDNA (prepared as described in Tarasova et al., J. Biol. Chem. 272: 14817-14824 (1997)) was performed as described (Tarasova et al., J Biol. Chem. 272(23): 14817-14824 (1997)). For the tests, the cells were seeded in 96-well plates at a density of 500-1000 cells per well and allowed to attach for 24 hours. The compounds were added to the cells at concentrations ranging from 10 pM to 10 μM and the cells were grown in a CO₂ incubator in the presence of compounds for 4-5 days. The cell number was estimated with either the methylthiazolyldiphenyl-tetrazolium bromide (MTT) assay (Alley et al., Cancer Res. 48(3): 589-601 (1988)) or sulforhodamine assay as described in Skehan et al., J. Natl. Cancer Inst. 82(13): 1107-1112 (1990)).

As shown in Table 1, administration of the conjugate to cells resulted in a dose-dependent decrease in the survival of only cells that expressed the gastrin receptor.

TABLE 1

| Concentration of | % Cell Survival | |
|---|---|---|
| Conjugate (μM) | (−) receptor expression | (+) receptor expression |
| 0.01 | 95 | 85 |
| 0.1 | 81 | 57 |
| 1 | 78 | 15 |
| 10 | 89 | 0 |

This example demonstrates that a cytotoxic drug can be delivered in a cell-specific manner.

### Example 8

This example demonstrates an assay to test the cytotoxicity of the conjugates in vivo.

Conjugates comprising cytotoxic drugs are tested for in vivo activity by performing assays that are described previously (See Dykes et al., Contrib. Oncol. 42: 1-22 (1992) and Plowman et al., Anti-cancer Drug Development Guide: Preclinical Screening, Clinical Trials and Approval, Humana Press: Totowa, N.J., 101-125 (1997)). Briefly, drug conjugates are injected into grafted tumors obtained from mice. The size of the injected tumor is measured 2-3 times/week and compared to the size of a control tumor.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A conjugate comprising a ligand, a linker, and a cytotoxic agent, in which the linker is FALA (SEQ ID NO: 1).

2. The conjugate of claim 1, wherein the ligand is a peptide or a peptidomimetic.

3. The conjugate of claim 2, wherein the peptidomimetic is a peptoid.

4. The conjugate of claim 1, wherein the ligand specifically binds to a receptor selected from the group consisting of:
the gastrin (cholecystokinin B (CCKB)) receptor,
the cholecystokinin A (CCKA) receptor,
the somatostatin receptor,
the gastrin-releasing peptide (GRP) receptor,
the substance P (neurokinin 1 (NK1)) receptor,
the guanylin receptor, and
the vasoactive intestinal peptide 1 (VIP-1) receptor.

5. The conjugate of claim 4, wherein the ligand is selected from the group consisting of:
LGPQGPPHLVADPSKKQGPWLEEEEEAYGWMDF (gastrin-34) (SEQ ID NO: 5),
an N-terminal truncated derivative of gastrin-34, and
W(Nle)DF (SEQ ID NO: 6).

6. The conjugate of claim 4, wherein the ligand is selected from the group consisting of:
D(SfY)MGWMDF (SEQ ID NO: 7),
D(SfY)(Nle)GW(Nle)DF (SEQ ID NO: 8), and
EEEAYGW(Nle)DF (SEQ ID NO:20).

7. The conjugate of claim 4, wherein the ligand is selected from the group consisting of:
VPLPAGGGTVLTKMYPRGNHWAVGHLM (SEQ ID NO: 9),
an N-terminal truncated derivative of VPLPAGGGTVLTKMYPRGNHWAVGHLM (SEQ ID NO: 9), and
WAVGHLM (SEQ ID NO: 10).

8. The conjugate of claim 4, wherein the ligand is selected from the group consisting of:
AGCKNFFWKTFTSC (SEQ ID NO: 11), in which the two C residues are disulfide bonded, and
FCFWKTCT(OH) (SEQ ID NO: 12), in which the two C residues are disulfide bonded.

9. The conjugate of claim 4, wherein the ligand is selected from the group consisting of:
RPLPQQFFGLM (SEQ ID NO: 13) and
an analog of RPLPQQFFGLM (SEQ ID NO: 13).

10. The conjugate of claim 4, wherein the ligand is selected from the group consisting of:
PGTCEICAYAACTGC (SEQ ID NO: 14), in which the first and third C residues are disulfide bonded, and
PGTCEICAYAACTGC (SEQ ID NO: 14), in which the second and fourth C residues are disulfide bonded.

11. The conjugate of claim 4, wherein the ligand is selected from the group consisting of:
NDDCELCVACTGCL (SEQ ID NO: 15), in which the first and third C residues are disulfide bonded, and
NDDCELCVACTGCL (SEQ ID NO: 15), in which the second and fourth C residues are disulfide bonded.

12. The conjugate of claim 4, wherein the ligand is selected from the group consisting of:
NYCCELCCNPACTGCF (SEQ ID NO: 16), in which the first and fourth C residues are disulfide bonded,
NYCCELCCNPACTGCF (SEQ ID NO: 16), in which the second and fifth C residues are disulfide bonded, and
NYCCELCCNPACTGCF (SEQ ID NO: 16), in which the third and sixth C residues are disulfide bonded.

13. The conjugate of claim 4, wherein the ligand is selected from the group consisting of:
HSDALFTDNYTRLRLQMAVKKYLNSILNG (SEQ ID NO: 17) and
HSDALFTDNYTRLRLQ(Nle)AVKKYLNSILNG (SEQ ID NO: 18).

14. The conjugate of claim 1, wherein the cytotoxic agent is selected from the group consisting of:
cemadotin,
a derivative of cemadotin,
a derivative of hemiasterlin,
esperamicin C,
neocarzinostatin,
maytansinoid DM1,
7-chloromethyl-10,11 methylenedioxy-camptothecin,
rhizoxin, and
the halichondrin B analog, ER-086526.

15. A conjugate comprising a ligand, a linker, and a cytotoxic agent, in which the linker is VLALA (SEQ ID NO: 2).

16. The conjugate of claim 15, wherein the ligand is a peptide or a peptidomimetic.

17. The conjugate of claim 16, wherein the peptidomimetic is a peptoid.

18. The conjugate of claim 15, wherein the ligand specifically binds to a receptor selected from the group consisting of:
the gastrin (CCKB) receptor,
the CCKA receptor,
the somatostatin receptor,
the GRP receptor,
the substance P (NK1) receptor,
the guanylin receptor, and
the VIP-1 receptor.

19. The conjugate of claim 18, wherein the ligand is selected from the group consisting of:
LGPQGPPHLVADPSKKQGPWLEEEEEAYGWMDF (gastrin-34) (SEQ ID NO: 5),
an N-terminal truncated derivative of gastrin-34, and
W(Nle)DF (SEQ ID NO: 6).

20. The conjugate of claim 18, wherein the ligand is selected from the group consisting of:
D(SfY)MGWMDF (SEQ ID NO: 7),
D(SfY)(Nle)GW(Nle)DF (SEQ ID NO: 8), and
EEEAYGW(Nle)DF (SEQ ID NO: 20).

21. The conjugate of claim 18, wherein the ligand is selected from the group consisting of:
VPLPAGGGTVLTKMYPRGNHWAVGHLM (SEQ ID NO: 9),
an N-terminal truncated derivative of VPLPAGGGTVLTKMYPRGNHWAVGHLM (SEQ ID NO: 9), and
WAVGHLM (SEQ ID NO: 10).

22. The conjugate of claim 18, wherein the ligand is selected from the group consisting of:
AGCKNFFWKTFTSC (SEQ ID NO: 11), in which the two C residues are disulfide bonded, and
FCFWKTCT(OH) (SEQ ID NO: 12), in which the two C residues are disulfide bonded.

23. The conjugate of claim 18, wherein the ligand is selected from the group consisting of:
RPLPQQFFGLM (SEQ ID NO: 13) and
an analog of RPLPQQFFGLM (SEQ ID NO: 13).

24. The conjugate of claim 18, wherein the ligand is selected from the group consisting of:
PGTCEICAYAACTGC (SEQ ID NO: 14), in which the first and third C residues are disulfide bonded, and
PGTCEICAYAACTGC (SEQ ID NO: 14), in which the second and fourth C residues are disulfide bonded.

25. The conjugate of claim 18, wherein the ligand is selected from the group consisting of:
NDDCELCVACTGCL (SEQ ID NO: 15), in which the first and third C residues are disulfide bonded, and
NDDCELCVACTGCL (SEQ ID NO: 15), in which the second and fourth C residues are disulfide bonded.

26. The conjugate of claim 18, wherein the ligand is selected from the group consisting of:
NYCCELCCNPACTGCF (SEQ ID NO: 16), in which the first and fourth C residues are disulfide bonded,
NYCCELCCNPACTGCF (SEQ ID NO: 16), in which the second and fifth C residues are disulfide bonded, and
NYCCELCCNPACTGCF (SEQ ID NO: 16), in which the third and sixth C residues are disulfide bonded.

27. The conjugate of claim 18, wherein the ligand is selected from the group consisting of:
HSDALFTDNYTRLRLQMAVKKYLNSILNG (SEQ ID NO: 17) and
HSDALFTDNYTRLRLQ(Nle)AVKKYLNSILNG (SEQ ID NO: 18).

28. The conjugate of claim 15, wherein the cytotoxic agent is selected from the group consisting of:
cemadotin,
a derivative of cemadotin,
a derivative of hemiasterlin,
esperamicin C,
neocarzinostatin,
maytansinoid DM1,
7-chloromethyl-10,11 methylenedioxy-camptothecin,
rhizoxin, and
the halichondrin B analog, ER-086526.

29-48. (canceled)

49. A conjugate comprising a ligand, a linker, and a cytotoxic agent, in which the linker is ChaLALA (SEQ ID NO: 21), ChaChaLAL (SEQ ID NO: 22), NalChaLAL (SEQ ID NO: 23) or NalLALA (SEQ ID NO: 24).

50. The conjugate of claim 49, wherein the ligand is a peptide or a peptidomimetic.

51. The conjugate of claim 50, wherein the peptidomimetic is a peptoid.

52. The conjugate of claim 49, wherein the ligand specifically binds to a receptor selected from the group consisting of:
the gastrin (cholecystokinin B (CCKB)) receptor,
the cholecystokinin A (CCKA) receptor,
the somatostatin receptor,
the gastrin-releasing peptide (GRP) receptor,
the substance P (neurokinin 1 (NK1)) receptor,
the guanylin receptor, and
the vasoactive intestinal peptide 1 (VIP-1) receptor.

53. The conjugate of claim 52, wherein the ligand is selected from the group consisting of:
LGPQGPPHLVADPSKKQGPWLEEEEEAYGWMDF (gastrin-34) (SEQ ID NO: 5),
an N-terminal truncated derivative of gastrin-34, and
W(Nle)DF (SEQ ID NO: 6).

54. The conjugate of claim 52, wherein the ligand is selected from the group consisting of:
D(SfY)MGWMDF (SEQ ID NO: 7),
D(SfY)(Nle)GW(Nle)DF (SEQ ID NO: 8), and
EEEAYGW(Nle)DF (SEQ ID NO:20).

55. The conjugate of claim 52, wherein the ligand is selected from the group consisting of:
VPLPAGGGTVLTKMYPRGNHWAVGHLM (SEQ ID NO: 9),
an N-terminal truncated derivative of VPLPAGGGTVLTKMYPRGNHWAVGHLM (SEQ ID NO: 9), and
WAVGHLM (SEQ ID NO: 10).

56. The conjugate of claim 52, wherein the ligand is selected from the group consisting of:
AGCKNFFWKTFTSC (SEQ ID NO: 11), in which the two C residues are disulfide bonded, and
FCFWKTCT(OH) (SEQ ID NO: 12), in which the two C residues are disulfide bonded.

57. The conjugate of claim 52, wherein the ligand is selected from the group consisting of:
RPLPQQFFGLM (SEQ ID NO: 13) and
an analog of RPLPQQFFGLM (SEQ ID NO: 13).

58. The conjugate of claim 52, wherein the ligand is selected from the group consisting of:
PGTCEICAYAACTGC (SEQ ID NO: 14), in which the first and third C residues are disulfide bonded, and
PGTCEICAYAACTGC (SEQ ID NO: 14), in which the second and fourth C residues are disulfide bonded.

59. The conjugate of claim 52, wherein the ligand is selected from the group consisting of:
NDDCELCVACTGCL (SEQ ID NO: 15), in which the first and third C residues are disulfide bonded, and
NDDCELCVACTGCL (SEQ ID NO: 15), in which the second and fourth C residues are disulfide bonded.

60. The conjugate of claim 52, wherein the ligand is selected from the group consisting of:
NYCCELCCNPACTGCF (SEQ ID NO: 16), in which the first and fourth C residues are disulfide bonded,
NYCCELCCNPACTGCF (SEQ ID NO: 16), in which the second and fifth C residues are disulfide bonded, and
NYCCELCCNPACTGCF (SEQ ID NO: 16), in which the third and sixth C residues are disulfide bonded.

61. The conjugate of claim 52, wherein the ligand is selected from the group consisting of:
HSDALFTDNYTRLRLQMAVKKYLNSILNG (SEQ ID NO: 17) and
HSDALFTDNYTRLRLQ(Nle)AVKKYLNSILNG (SEQ ID NO: 18).

62. The conjugate of claim 49, wherein the cytotoxic agent is selected from the group consisting of:
cemadotin,
a derivative of cemadotin,
a derivative of hemiasterlin,
esperamicin C,
neocarzinostatin,
maytansinoid DM1,
7-chloromethyl-10,11 methylenedioxy-camptothecin,
rhizoxin, and
the halichondrin B analog, ER-086526.

63. A composition comprising the conjugate of claim 1 and a carrier.

64. A composition comprising the conjugate of claim 15 and a carrier.

65. (canceled)

66. (canceled)

67. A composition comprising the conjugate of claim 49 and a carrier

68. A method of delivering a cytotoxic agent in a cell-specific manner, which method comprises administering the conjugate of claim 1 to a collection of cells comprising a receptor to which the ligand of the conjugate binds, whereupon the cytotoxic agent is administered to the cells in a cell-specific manner.

69. The method of claim 68, wherein the cells are in vivo.

70. A method of delivering a cytotoxic agent in a cell-specific manner, which method comprises administering the conjugate of claim 15 to a collection of cells comprising a receptor to which the ligand of the conjugate binds, whereupon the cytotoxic agent is administered to the cells in a cell-specific manner.

71. The method of claim 70, wherein the cells are in vivo.

72-75. (canceled)

76. A method of delivering a cytotoxic agent in a cell-specific manner, which method comprises administering the conjugate of claim 49 to a collection of cells comprising a receptor to which the ligand of the conjugate binds, whereupon the cytotoxic agent is administered to the cells in a cell-specific manner.

77. The method of claim 76, wherein the cells are in vivo.

78. A method of treating cancer in a mammal, which method comprises administering a cancer-treating effective amount of the conjugate of claim 1 to the mammal, whereupon the mammal is treated for cancer.

79. The method of claim 78, wherein the cancer is cancer of the lung, stomach, colon, breast, or pancreas.

80. A method of treating cancer in a mammal, which method comprises administering a cancer-treating effective amount of the conjugate of claim 15 to the mammal, whereupon the mammal is treated for cancer.

81. The method of claim 80, wherein the cancer is cancer of the lung, stomach, colon, breast, or pancreas.

82-85. (canceled)

86. A method of treating cancer in a mammal, which method comprises administering a cancer-treating effective amount of the conjugate of claim 49 to the mammal, whereupon the mammal is treated for cancer.

87. The method of claim 86, wherein the cancer is cancer of the lung, stomach, colon, breast, or pancreas.

88. A conjugate comprising a ligand, a linker and a cytotoxic agents, in which the linker is ALAL (SEQ ID NO: 3) and the ligand specifically binds to a receptor selected from the group consisting of:
the gastrin (cholecystokinin B (CCKB)) receptor,
the cholecystokinin A (CCKA) receptor,
the somatostatin receptor,
the gastrin-releasing peptide (GRP) receptor,
the substance P (neurokinin 1 (NK1)) receptor,
the guanylin receptor, and
the vasoactive intestinal peptide 1 (VIP-1) receptor.

89. The conjugate of claim 88, wherein the ligand is selected from the group consisting of:
LGPQGPPHLVADPSKKQGPWLEEEEEAYGWMDF (gastrin-34) (SEQ ID NO: 5),
an N-terminal truncated derivative of gastrin-34, and
W(Nle)DF (SEQ ID NO: 6).

90. The conjugate of claim 88, wherein the ligand is selected from the group consisting of:
D(SfY)MGWMDF (SEQ ID NO: 7),
D(SfY)(Nle)GW(Nle)DF (SEQ ID NO: 8), and
EEEAYGW(Nle)DF (SEQ ID NO: 20).

91. The conjugate of claim 88, wherein the ligand is selected from the group consisting of:
VPLPAGGGTVLTKMYPRGNHWAVGHLM (SEQ ID NO: 9),
an N-terminal truncated derivative of VPLPAGGGTVLTKMYPRGNHWAVGHLM (SEQ ID NO: 9), and
WAVGHLM (SEQ ID NO: 10).

92. The conjugate of claim 88, wherein the ligand is selected from the group consisting of:
AGCKNFFWKTFTSC (SEQ ID NO: 11), in which the two C residues are disulfide bonded, and
FCFWKTCT(OH) (SEQ ID NO: 12), in which the two C residues are disulfide bonded.

93. The conjugate of claim 88, wherein the ligand is selected from the group consisting of:
RPLPQQFFGLM (SEQ ID NO: 13) and
an analog of RPLPQQFFGLM (SEQ ID NO: 13).

94. The conjugate of claim 88, wherein the ligand is selected from the group consisting of:
PGTCEICAYAACTGC (SEQ ID NO: 14), in which the first and third C residues are disulfide bonded, and
PGTCEICAYAACTGC (SEQ ID NO: 14), in which the second and fourth C residues are disulfide bonded.

95. The conjugate of claim 88, wherein the ligand is selected from the group consisting of:
NDDCELCVACTGCL (SEQ ID NO: 15), in which the first and third C residues are disulfide bonded, and
NDDCELCVACTGCL (SEQ ID NO: 15), in which the second and fourth C residues are disulfide bonded.

96. The conjugate of claim 88, wherein the ligand is selected from the group consisting of:
NYCCELCCNPACTGCF (SEQ ID NO: 16), in which the first and fourth C residues are disulfide bonded,
NYCCELCCNPACTGCF (SEQ ID NO: 16), in which the second and fifth C residues are disulfide bonded, and
NYCCELCCNPACTGCF (SEQ ID NO: 16), in which the third and sixth C residues are disulfide bonded.

97. The conjugate of claim 88, wherein the ligand is selected from the group consisting of:
HSDALFTDNYTRLRLQMAVKKYLNSILNG (SEQ ID NO: 17) and
HSDALFTDNYTRLRLQ(Nle)AVKKYLNSILNG (SEQ ID NO: 18).

98. The conjugate of claim 88, wherein the cytotoxic agent, is selected from the group consisting of:
cemadotin,
a derivative of cemadotin,
a derivative of hemiasterlin,
esperamicin C,
neocarzinostatin,
maytansinoid DM1,
7-chloromethyl-10,11 methylenedioxy-camptothecin,
rhizoxin, and
the halichondrin B analog, ER-086526.

99. A conjugate comprising a ligand, a linker, and a cytotoxic agent, in which the linker is ALALA (SEQ ID NO: 4), wherein the ligand specifically binds to a receptor selected from the group consisting of:
the gastrin (cholecystokinin B (CCKB)) receptor,
the cholecystokinin A (CCKA) receptor,
the somatostatin receptor,
the gastrin-releasing peptide (GRP) receptor,
the substance P (neurokinin 1 (NK1)) receptor,
the guanylin receptor, and
the vasoactive intestinal peptide 1 (VIP-1) receptor.

100. The conjugate of claim 99, wherein the ligand is selected from the group consisting of:
LGPQGPPHLVADPSKKQGPWLEEEEEAYGWMDF (gastrin-34) (SEQ ID NO: 5), an N-terminal truncated derivative of gastrin-34, provided that the derivative is not
AYGW(Nle)DF (SEQ ID NO: 19), and
W(Nle)DF (SEQ ID NO: 6).

101. The conjugate of claim 99, wherein the ligand is selected from the group consisting of:
D(SfY)MGWMDF (SEQ ID NO: 7),
D(SfY)(Nle)GW(Nle)DF (SEQ ID NO: 8), and
EEEAYGW(Nle)DF (SEQ ID NO: 20).

102. The conjugate of claim 99, wherein the ligand is selected from the group consisting of:
VPLPAGGGTVLTKMYPRGNHWAVGHLM (SEQ ID NO: 9),
an N-terminal truncated derivative of VPLPAGGGTVLTKMYPRGNHWAVGHLM (SEQ ID NO: 9), and
WAVGHLM (SEQ ID NO: 10).

103. The conjugate of claim 99, wherein the ligand is selected from the group consisting of:
AGCKNFFWKTFTSC (SEQ ID NO: 11), in which the two C residues are disulfide bonded, and
FCFWKTCT(OH) (SEQ ID NO: 12), in which the two C residues are disulfide bonded.

104. The conjugate of claim 99, wherein the ligand is selected from the group consisting of:
RPLPQQFFGLM (SEQ ID NO: 13) and
an analog of RPLPQQFFGLM (SEQ ID NO: 13).

105. The conjugate of claim 99, wherein the ligand is selected from the group consisting of:
PGTCEICAYAACTGC (SEQ ID NO: 14), in which the first and third C residues are disulfide bonded, and
PGTCEICAYAACTGC (SEQ ID NO: 14), in which the second and fourth C residues are disulfide bonded.

106. The conjugate of claim 99, wherein the ligand is selected from the group consisting of:
NDDCELCVACTGCL (SEQ ID NO: 15), in which the first and third C residues are disulfide bonded, and
NDDCELCVACTGCL (SEQ ID NO: 15), in which the second and fourth C residues are disulfide bonded.

107. The conjugate of claim 99, wherein the ligand is selected from the group consisting of:
NYCCELCCNPACTGCF (SEQ ID NO: 16), in which the first and fourth C residues are disulfide bonded,
NYCCELCCNPACTGCF (SEQ ID NO: 16), in which the second and fifth C residues are disulfide bonded, and
NYCCELCCNPACTGCF (SEQ ID NO: 16), in which the third and sixth C residues are disulfide bonded.

108. The conjugate of claim 99, wherein the ligand is selected from the group consisting of:
HSDALFTDNYTRLRLQMAVKKYLNSILNG (SEQ ID NO: 17) and
HSDALFTDNYTRLRLQ(Nle)AVKKYLNSILNG (SEQ ID NO: 18).

109. The conjugate of claim 99, wherein the cytotoxic agent is selected from the group consisting of:
cemadotin,
a derivative of cemadotin,
a derivative of hemiasterlin,
esperamicin C,
neocarzinostatin,
maytansinoid DM1,
7-chloromethyl-10,11 methylenedioxy-camptothecin,
rhizoxin, and
the halichondrin B analog, ER-086526.

110. A composition comprising the conjugate of claim 88 and a carrier.

111. A composition comprising the conjugate of claim 99 and a carrier.

112. A method of delivering a cytotoxic agent in a cell-specific manner, which method comprises administering the conjugate of claim 88 to a collection of cells comprising a receptor to which the ligand of the conjugate binds, whereupon the cytotoxic agent is administered to the cells in a cell-specific manner.

113. The method of claim 112, wherein the cells are in vivo.

114. A method of delivering a cytotoxic agent in a cell-specific manner, which method comprises administering the conjugate of claim 99 to a collection of cells comprising a receptor to which the ligand of the conjugate binds, whereupon the cytotoxic agent is administered to the cells in a cell-specific manner.

115. The method of claim 114, wherein the cells are in vivo.

116. A method of treating cancer in a mammal, which method comprises administering a cancer-treating effective amount of the conjugate of claim 88 to the mammal, whereupon the mammal is treated for cancer.

117. The method of claim 116, wherein the cancer is cancer of the lung, stomach, colon, breast, or pancreas.

118. A method of treating cancer in a mammal, which method comprises administering a cancer-treating effective amount of the conjugate of claim 99 to the mammal, whereupon the mammal is treated for cancer.

119. The method of claim 118, wherein the cancer is cancer of the lung, stomach, colon, breast, or pancreas.
